(19) United States
(12) Patent Application Publication
(10) Pub. No.: US 2023/0090391 A1
(43) Pub. Date: Mar. 23, 2023

(54) OMECAMTIV MECARBIL TABLET

(71) Applicant: Amgen Inc., THOUSAND OAKS, CA (US)

(72) Inventors: Mingda BI, Simi Valley, CA (US); Yuan-Hon KIANG, Thousand Oaks, CA (US); Hao LOU, Thousand Oaks, CA (US)

(21) Appl. No.: 17/798,531

(22) PCT Filed: Feb. 10, 2021
(86) PCT No.: PCT/US2021/017429

§ 371 Date: Aug. 9, 2022

Related U.S. Application Data
(60) Provisional application No. 62/972,506, filed on Feb. 10, 2020.

(51) Int. Cl. A61K 31/496; A61K 9/28; A61K 9/20

(52) U.S. Cl. CPC A61K 31/496; A61K 9/2893; A61K 9/2095; A61K 9/2866; A61K 9/2853; A61K 9/2018; A61K 9/2054; A61K 9/2009; A61K 9/2013

(57) ABSTRACT

Provided herein are tablet formulations comprising a core comprising omecamtiv mecarbil, a pharmaceutically acceptable salt thereof, or a pharmaceutically acceptable hydrate of a pharmaceutically acceptable salt thereof; a filler; a binder; a glidant; and a lubricant; and a film coating on the core, the film coating comprising a modified-release polymer and a pore former. Also provided is a process for making a tablet formulation and a method of treating cardiovascular conditions, such as heart failure, using the tablet formulation.

### BACKGROUND

Omecamtiv mecarbil (OM) is an activator of cardiac myosin that directly targets the contractile mechanisms of cardiac myocytes intended to enhance efficiency of myocardial contraction in patients suffering from a cardiovascular condition, such as heart failure. OM is currently in Phase 3 clinical trials. A modified-release (MR) tablet of OM for oral administration having dimensions of 14.4 mm×8.4 mm was developed for adult patients (see International Patent Application Publication WO2014/152236A1) to reduce the maximum plasma concentration (Cₘₐₓ) and to safely provide an efficient amount of OM to patients. To ensure patient compliance, however, a need exists for an OM formulation that is easy to swallow and administer and suitable for use in certain patient populations, such as pediatric patients (for example, 6-18 years of age) and adult patients with difficulty swallowing.

### SUMMARY

Provided herein are tablet formulations comprising a core comprising omecamtiv mecarbil, a pharmaceutically acceptable salt thereof, or a pharmaceutically acceptable hydrate of a pharmaceutically acceptable salt thereof; a filler; a binder; a glidant; and a lubricant; and a film coating on the core, the film coating comprising a modified-release polymer and a pore former.

Also provided herein are processes for making the disclosed tablet formulations and using the disclosed tablet formulations to treat patients suffering from a cardiovascular condition, such as heart failure.

### BRIEF DESCRIPTION OF THE FIGURES

FIG. 1 shows the dissolution profiles of omecamtiv mecarbil dihydrochloride monohydrate modified-release tablets in pH 6.8 phosphate buffer.

FIG. 2 shows the manufacturing flow diagram for omecamtiv mecarbil dihydrochloride monohydrate 1 mg immediate-release mini-tablet cores.

FIG. 3 shows an overlay of ¹⁹F solid state nuclear magnetic resonance (SSNMR) spectra of omecamtiv mecarbil granulation.

FIG. 4 shows an overlay of ¹⁹F SSNMR spectra of omecamtiv mecarbil immediate-release mini-tablet cores.

FIG. 5 shows the manufacturing flow diagram for modified-release coating of omecamtiv mecarbil dihydrochloride monohydrate 1 mg immediate release mini-tablet cores.

FIG. 6 shows the dissolution profiles of omecamtiv mecarbil dihydrochloride monohydrate 1 mg modified-release mini-tablets (n=3) coated with 70:30 CA:PEG to 10% coating weight gain in pH 6.8 buffer.

FIG. 7 shows the dissolution profiles of omecamtiv mecarbil dihydrochloride monohydrate 1 mg modified-release mini-tablets (n=3) coated with 70:30 CA:PEG to 15% coating weight gain in pH 6.8 buffer.

FIG. 8 shows the dissolution profiles of omecamtiv mecarbil dihydrochloride monohydrate 1 mg modified-release mini-tablets (n=3) coated with 70:30 CA:PEG to 20% coating weight gain in pH 6.8 buffer.

FIG. 9 shows the dissolution profiles of omecamtiv mecarbil dihydrochloride monohydrate 1 mg modified-release mini-tablets (n=3) coated with 70:30 CA:PEG or 50:50 CA:PEG to 10% coating weight gain in pH 6.8 buffer.

FIG. 10 shows the manufacturing flow diagram for modified-release coating of omecamtiv mecarbil dihydrochloride monohydrate 1 mg immediate-release mini-tablet cores.

FIG. 11 shows a comparison of dissolution profiles for omecamtiv mecarbil dihydrochloride monohydrate 1 mg modified-release mini-tablets (n=6) between batches (USP II, 50 mM pH 6.8 phosphate buffer, 500 mL, 75 RPM, 37° C.).

FIG. 12 shows a comparison of dissolution profiles for omecamtiv mecarbil dihydrochloride monohydrate 1 mg modified-release mini-tablets (n=6) stored for one month at 25° C./60% RH (USP II, 50 mM pH 6.8 phosphate buffer, 500 mL, 75 RPM, 37° C.).

FIG. 13 shows an X-ray powder diffraction pattern (XRPD) for Form A of omecamtiv mecarbil dihydrochloride monohydrate.

FIG. 14 shows release profiles of omecamtiv mecarbil from disclosed tablet formulations.

FIG. 15A shows plasma concentration profiles (arithmetic mean) for omecamtiv mecarbil mini-tablet formulations (25×1 mg) through 168 hours (linear scale).

FIG. 15B shows plasma concentration profiles (arithmetic mean) for omecamtiv mecarbil mini-tablet formulations (25×1 mg) through 168 hours (semi-logarithmic scale).

FIG. 16A shows plasma concentration profiles (arithmetic mean) for omecamtiv mecarbil mini-tablet formulations (25×1 mg) through 72 hours (linear scale).

FIG. 16B shows plasma concentration profiles (arithmetic mean) for omecamtiv mecarbil mini-tablet formulations (25×1 mg) through 72 hours (semi-logarithmic scale).

### DETAILED DESCRIPTION

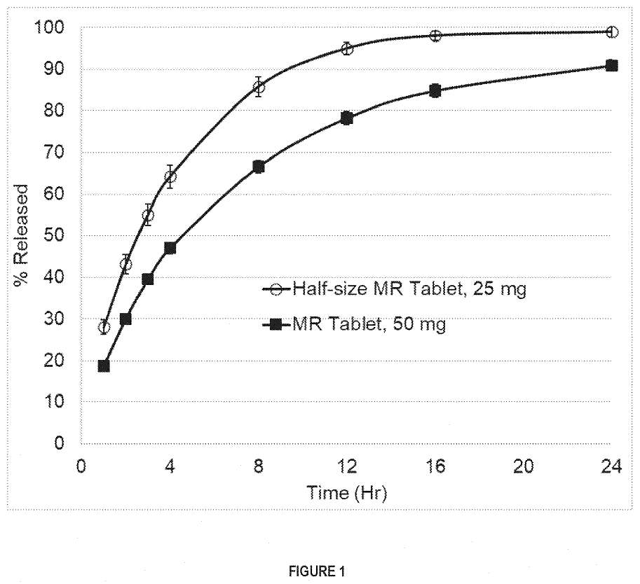

Prior to formulations disclosed herein, a number of conventional formulations were investigated in an effort to obtain an omecamtiv mecarbil formulation suitable for pediatric patients (for example, 6-18 years old, or 6-12 years old) or adult patients with difficulty in swallowing. For example, tablets having a reduced size were evaluated. However, as depicted in FIG. 1, simply reducing by half the physical size and dose of a modified-release (MR) tablet comprising omecamtiv mecarbil dihydrochloride monohydrate resulted in a significant undesirable increase in the dissolution rate of omecamtiv mecarbil, which can lead to an undesirable higher Cₘₐₓ. A higher Cₘₐₓ may lead to increased adverse events.

A tablet formulation for pediatric patients or adults with difficulty swallowing should exhibit physical properties (for example, shape and size) that would facilitate patient compliance (for example, swallowability) and exhibit suitable pharmacokinetic performance (for example, suitable Cₘₐₓ and reproducible release rate). Desirable attributes of the tablets include, but are not limited to, one or more of the following: a core having a round and biconvex shape; less than 3 mm in diameter and height; approximately 12.5 mg weight; a dosage strength of 1-3 mg omecamtiv mecarbil wherein the release from the core is independent of pH and 100% of omecamtiv mecarbil is released within 60 minutes. The MR coating can provide a release rate that is independent of pH and is tunable depending on the nature of the coating.

Provided herein are tablet formulations comprising a core having a film coating on the core, wherein the film coating comprises a modified-release polymer and a pore former. The core comprises omecamtiv mecarbil, a pharmaceutically acceptable salt thereof, or a pharmaceutically acceptable hydrate of a pharmaceutically acceptable salt thereof, and the uncoated core provides an immediate-release of omecamtiv mecarbil, a pharmaceutically acceptable salt thereof, or a pharmaceutically acceptable hydrate of a pharmaceutically acceptable salt thereof. The core comprises intra- and extra-granular components. The intra-granular components comprise omecamtiv mecarbil, a pharmaceutically acceptable salt thereof, or a pharmaceutically acceptable hydrate of a pharmaceutically acceptable salt thereof, as the active ingredient, one or more fillers, and one or more binders. The extra-granular components comprise one or more glidants and one or more lubricants. The intra- and extra-granular components can be formed into a core using a suitable method as described herein. The core can then be coated with the film coating using a suitable method to provide the disclosed tablet formulations.

The disclosed cores of the tablet formulations comprise omecamtiv mecarbil, a pharmaceutically acceptable salt thereof, or a pharmaceutically acceptable hydrate of a pharmaceutically acceptable salt thereof; a filler; a binder; a glidant; and a lubricant. The film coating on the core of the disclosed tablet formulations comprises a modified-release polymer (sometimes referred to as a control-release agent) and a pore former. In some embodiments, for any of the component amounts disclosed herein, a stated amount or weight percentage of the component can vary ±5%.

In some embodiments, the tablet formulation has a form or dimension(s) suitable for the intended patient population (for example, a pediatric patient). Accordingly, the tablet formulation can have a diameter of 5 mm or less, for example, 4.5 mm or less, 4 mm or less, or 3.5 mm or less. Alternatively, or in addition, the tablet formulation can have a diameter of 0.5 mm or more, for example, 1 mm or more, 1.5 mm or more, 2 mm or more, 2.5 mm or more, or 3 mm or more. Thus, the tablet formulation can have a diameter bounded by any of the aforementioned endpoints. For example, the tablet formulation can have a diameter of 0.5-5 mm, 1-4.5 mm, 1.5-4 mm, 2-3.5 mm, or 2.5-3 mm.

In some embodiments, the tablet formulation has a diameter of up to 3 mm (for example, 0.5 mm, 1 mm, 1.5 mm, 2 mm, 2.5 mm, or 3 mm).

The formulations disclosed herein can provide a desirable release profile of omecamtiv mecarbil. The release, or dissolution, profile of the formulation can be determined using any suitable method. An illustrative method is the U.S. Pharmacopeia (USP) II method using the following parameters: apparatus is USP <711> Apparatus II (paddle); vessel size/type is a 1000 mL clear glass, round bottom; rotation speed is 75 rpm; media volume is 500 mL; test temperature is 37.0±0.5° C.; dissolution media is phosphate buffer (pH 6.8); and sampling time points are 1, 2, 3, 4, 6, 8, 12, 16, and 24 hours. The test solutions are assayed using high performance liquid chromatography (HPLC) using the following conditions: pump is isocratic; reverse-phase column (for example, X-Bridge, 150×3 mm (id), C18, 3.5 μm particle size, commercially available from Waters); UV detection (235 nm); injection volume of 75 μL; flow rate of 0.5 mL/min; column temperature is 30° C.; autosampler temperature is ambient temperature; and a run time of 6 minutes.

In some embodiments, the tablet formulation releases up to 50% omecamtiv mecarbil at 1 hour, for example, up to 45%, up to 40%, up to 35%, up to 30%, up to 25%, up to 20%, up to 15%, or releases up to 10% omecamtiv mecarbil at 1 hour. In some embodiments, the tablet formulation releases 5-10% omecamtiv mecarbil at 1 hour, for example, 7% omecamtiv mecarbil at 1 hour. In some embodiments, the tablet formulation releases 15-25% omecamtiv mecarbil at 1 hour, for example, 20% omecamtiv mecarbil at 1 hour. In some embodiments, the tablet formulation releases 35-45% omecamtiv mecarbil at 1 hour, for example, 41% omecamtiv mecarbil at 1 hour.

In some embodiments, the tablet formulation releases up to 70% omecamtiv mecarbil at 2 hours, for example, up to 65%, up to 60%, up to 55%, up to 50%, up to 45%, up to 40%, up to 35%, or releases up to 30% omecamtiv mecarbil at 2 hours. In some embodiments, the tablet formulation releases 25-35% omecamtiv mecarbil at 2 hours, for example, 28% omecamtiv mecarbil at 2 hours. In some embodiments, the tablet formulation releases 35-45% omecamtiv mecarbil at 2 hours, for example, 40% omecamtiv mecarbil at 2 hours. In some embodiments, the tablet formulation releases 60-70% omecamtiv mecarbil at 2 hours, for example, 66% omecamtiv mecarbil at 2 hours.

In some embodiments, the tablet formulation releases up to 90% omecamtiv mecarbil at 8 hours, for example, up to 85%, up to 80%, or releases up to 75% omecamtiv mecarbil at 8 hours. In some embodiments, the tablet formulation releases 70-75% omecamtiv mecarbil at 8 hours, for example, 72% omecamtiv mecarbil at 8 hours. In some embodiments, the tablet formulation releases 75-80% omecamtiv mecarbil at 8 hours, for example, 77% omecamtiv mecarbil at 8 hours. In some embodiments, the tablet formulation releases 85-90% omecamtiv mecarbil at 8 hours, for example, 87% omecamtiv mecarbil at 8 hours.

In some embodiments, the tablet formulation releases up to 95% omecamtiv mecarbil at 16 hours, for example, up to 90%, up to 85%, or releases up to 80% omecamtiv mecarbil at 16 hours. In some embodiments, the tablet formulation releases 78-83% omecamtiv mecarbil at 16 hours for example, 81% omecamtiv mecarbil at 16 hours. In some embodiments, the tablet formulation releases 85-90% omecamtiv mecarbil at 16 hours, for example, 86% omecamtiv mecarbil at 16 hours. In some embodiments, the tablet formulation releases 90-95% omecamtiv mecarbil at 16 hours, for example, 93% omecamtiv mecarbil at 16 hours.

In some embodiments, the tablet formulation provides an omecamtiv mecarbil release profile of less than or equal to 50% omecamtiv mecarbil released at 1 hour; 60-70% omecamtiv mecarbil released at 2 hours; 85-90% omecamtiv mecarbil released at 8 hours; and greater than or equal to 90% omecamtiv mecarbil released at 16 hours.

In some embodiments, the tablet formulation provides an omecamtiv mecarbil release profile of less than or equal to 25% omecamtiv mecarbil released at 1 hour; 35-45% omecamtiv mecarbil released at 2 hours; 75-80% omecamtiv mecarbil released at 8 hours; and greater than or equal to 85% omecamtiv mecarbil released at 16 hours.

In some embodiments, the tablet formulation provides an omecamtiv mecarbil release profile of less than or equal to 10% omecamtiv mecarbil released at 1 hour; 25-35% omecamtiv mecarbil released at 2 hours; 70-75% omecamtiv mecarbil released at 8 hours; and greater than or equal to 78% omecamtiv mecarbil released at 16 hours.

The tablet formulation provides a suitable Cₘₐₓ of omecamtiv mecarbil upon administration to a patient. In some embodiments, the tablet formulation provides a Cₘₐₓ of omecamtiv mecarbil of 100 ng/mL or more upon administration to a patient, for example, 125 ng/mL or more, 150 ng/mL or more, 175 ng/mL or more, 200 ng/mL or more, 225 ng/mL or more, 250 ng/mL or more, 275 ng/mL or more, 300 ng/mL or more, 325 ng/mL or more, 350 ng/mL or more, 375 ng/mL or more, 400 ng/mL or more, 425 ng/mL or more, 450 ng/mL or more, 475 ng/mL or more, or a Cₘₐₓ of omecamtiv mecarbil of 500 ng/mL or more upon administration to a patient. Alternatively, or in addition, the tablet formulation provides a Cₘₐₓ of omecamtiv mecarbil of 1000 ng/mL or less upon administration to a patient, for example, 975 ng/mL or less, 950 ng/mL or less, 925 ng/mL or less, 900 ng/mL or less, 875 ng/mL or less, 850 ng/mL or less, 825 ng/mL or less, 800 ng/mL or less, 775 ng/mL or less, 750 ng/mL or less, 725 ng/mL or less, 700 ng/mL or less, 675 ng/mL or less, 650 ng/mL or less, 625 ng/mL or less, 600 ng/mL or less, 575 ng/mL or less, 550 ng/mL or less, or a Cₘₐₓ of omecamtiv mecarbil of 525 ng/mL or less upon administration to a patient.

Thus, the tablet formulation can provide a Cₘₐₓ of omecamtiv mecarbil to a patient bounded by any two of the aforementioned endpoints. For example, the tablet formulation can provide a Cₘₐₓ of omecamtiv mecarbil of 100-1000 ng/mL, 125-975 ng/mL, 150-950 ng/mL, 175-925 ng/mL, 200-900 ng/mL, 225-875 ng/mL, 250-850 ng/mL, 275-825 ng/mL, 300-800 ng/mL, 325-775 ng/mL, 350-750 ng/mL, 375-725 ng/mL, 400-700 ng/mL, 425-675 ng/mL, 450-650 ng/mL, 475-625 ng/mL, 500-600 ng/mL, or a Cₘₐₓ of omecamtiv mecarbil of 525-575 ng/mL upon administration to a patient.

In some embodiments, the tablet formulation, upon administration to a patient, provides a Cₘₐₓ of omecamtiv mecarbil of 100-1000 ng/mL or 300-1000 ng/mL.

### Omecamtiv Mecarbil

Omecamtiv mecarbil (AMG 423, CK-1827452) has the structure:

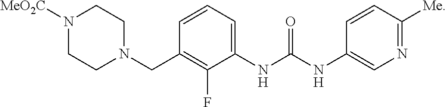 [SMILES: COC(=O)N1CCN(Cc2cccc(NC(=O)Nc3ccc(C)nc3)c2F)CC1]

(see, for example, International Patent Application Publication WO2014/152236A1, paragraph [0005])

The omecamtiv mecarbil used in the disclosed formulations can be present as omecamtiv mecarbil free base or pharmaceutically acceptable salt or hydrate of a pharmaceutically acceptable salt thereof.

“Pharmaceutically acceptable salts” include, but are not limited to (1) acid addition salts, (a) formed with inorganic acids, such as hydrochlorate (i.e., hydrochloride), phosphate, diphosphate, hydrobromate, sulfate, sulfinate, nitrate, and like salts; or (b) formed with an organic acid, such as malate, maleate, fumarate, tartrate, succinate, citrate, acetate, lactate, methanesulfonate, p-toluenesulfonate, 2-hydroxyethylsulfonate, benzoate, salicylate, stearate, and alkanoate such as acetate, HOOC—(CH₂)ₙ—COOH where n is 0-4, and like salts; and (2) salts formed when an acidic proton of omecamtiv mercarbil is replaced by a pharmaceutically acceptable cation including, but are not limited to sodium, potassium, calcium, aluminum, lithium, and ammonium. Those skilled in the art will recognize various synthetic methodologies that may be used to prepare non-toxic pharmaceutically acceptable addition salts.

In some cases, tablet formulation comprises omecamtiv mecarbil. In one embodiment, the tablet formulation comprises omecamtiv mecarbil dihydrochloride. In a further embodiment, the tablet formulation comprises omecamtiv mecarbil dihydrochloride monohydrate. In yet another embodiment, the tablet formulation comprises omecamtiv mecarbil dihydrochloride monohydrate Form A, as disclosed in International Patent Application Publication No. WO2014/152270A1.

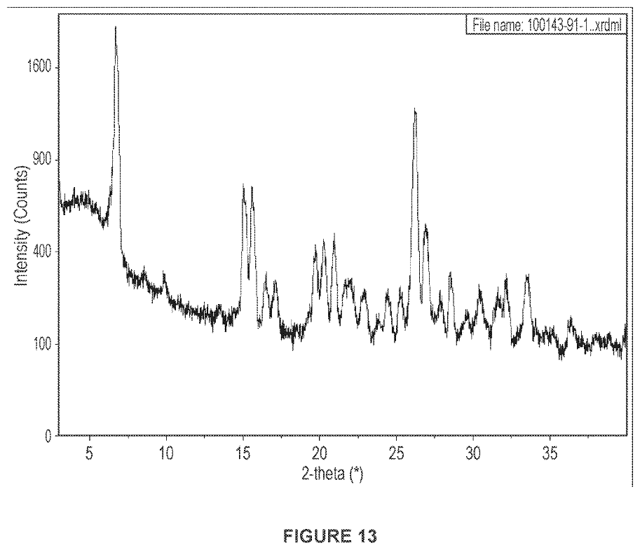

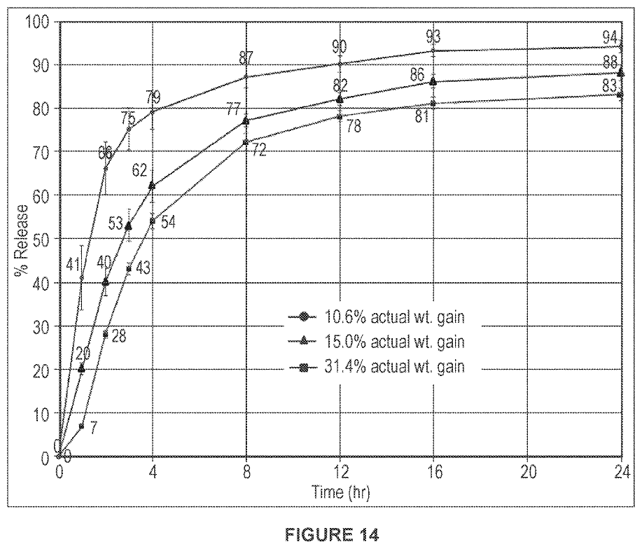

Form A can be characterized by an X-ray powder diffraction (XRPD) pattern, obtained as set forth in WO2014/152270A1, having peaks at 6.6, 14.9, 20.1, 21.4, and 26.8±0.2° 2θ using Cu Kα radiation. Form A optionally can be further characterized by an XRPD pattern having additional peaks at 8.4, 24.2, 26.0, 33.3±0.2° 2θ using Cu Kα radiation. Form A optionally can be even further characterized by an XRPD pattern having additional peaks at 6.2, 9.7, 13.2, 14.3, 15.4, 16.3, 16.9, 18.9, 19.5, 20.7, 21.8, 22.8, 23.6, 25.1, 27.3, 27.7, 28.4, 29.4, 30.2, 31.2, 31.5, 31.9, 33.9, 34.5, 34.9, 36.1, 36.8, 37.7, 38.5, and 39.7 0.2° 2θ using Cu Kα radiation. In various cases, Form A can be characterized by an XRPD pattern having peaks at 6.2, 6.6, 8.4, 9.7, 13.2, 14.3, 14.9, 15.4, 16.3, 16.9, 18.9, 19.5, 20.1, 20.7, 21.4, 21.8, 22.8, 23.6, 24.3, 25.1, 26.0, 26.8, 27.3, 27.7, 28.4, 29.4, 30.2, 31.2, 31.5, 31.9, 33.3, 33.9, 34.5, 34.9, 36.1, 36.8, 37.7, 38.5, and 39.7 0.2° 2θ using Cu Kα radiation. In some embodiments, Form A can be characterized by an X-ray powder diffraction pattern substantially as depicted in FIG. 13 wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

In some embodiments, the tablet formulation comprises omecamtiv mecarbil dihydrochloride Form B. In some embodiments, the tablet formulation comprises omecamtiv mecarbil dihydrochloride Form C. Form B and Form C polymorphs of omecamtiv mecarbil, are metastable anhydrous dihydrochloride forms, and can be formed under varied conditions and temperatures, as noted in WO2014/152270A1.

As described in WO2014/152270A1, Form B can be characterized by an XRPD pattern having peaks at 6.8, 8.8, 14.7, 17.7, and 22.3±0.2° 2θ using Cu Kα radiation. Form B optionally can be further characterized by an XRPD pattern having additional peaks at 9.6, 13.5, 19.2, 26.2±0.2° 2θ using Cu Kα radiation. Form B can be characterized by an XRPD pattern having peaks at 6.2, 6.8, 8.8, 9.6, 13.5, 14.4, 14.7, 15.4, 16.3, 17.0, 17.7, 18.3, 19.2, 19.9, 20.5, 20.8, 21.8, 22.3, 22.7, 23.0, 24.8, 25.1, 25.5, 26.2, 26.4, 26.8, 27.5, 28.5, 30.2, 30.6, 31.1, 31.5, 32.1, 32.7, 34.1, 34.4, 35.5, 35.9, 38.1, 38.9 0.2° 2θ using Cu Kα radiation. In some embodiments, Form B can be characterized by an XRPD pattern substantially as depicted in WO2014/152270A1, wherein by “substantially” is meant that the reported peaks can vary by ±0.2°.

As described in WO2014/152270A1, Form C can be characterized by an XRPD pattern having peaks at 6.7, 14.8, 17.4, 20.6, and 26.2±0.2° 2θ using Cu Kα radiation. Form C optionally can be further characterized by an XRPD pattern having additional peaks at 8.7, 22.0, 27.1, and 27.7±0.2° 2θ using Cu Kα radiation. Form C can be characterized by an XRPD pattern having peaks at 6.2, 6.7, 8.7, 9.6, 13.5, 14.5, 14.8, 15.4, 16.4, 17.1, 17.4, 18.4, 19.3, 19.5, 19.9, 20.6, 20.8, 21.8, 22.0, 22.5, 22.8, 24.3, 24.7, 25.1, 25.6, 26.2, 26.5, 27.1, 27.3, 27.7, 28.5, 30.0, 30.5, 31.0, 31.5, 32.2, 32.8, 34.1, 35.2, 36.0, 36.9, and 38.8±0.2° 2θ using Cu Kα radiation. In some embodiments, Form C can be characterized by an XRPD pattern substantially as depicted in WO2014/152270A1, wherein by “substantially” is meant that the reported peaks can vary by ±0.2°.

The tablet formulation comprises any suitable amount of omecamtiv mecarbil, a pharmaceutically acceptable salt thereof, or a pharmaceutically acceptable hydrate of a pharmaceutically acceptable salt thereof. If the tablet formulation comprises too little omecamtiv mecarbil, the pill burden to the patient will be unduly increased. In contrast, if the tablet formulation comprises too much omecamtiv mecarbil, the tablet formulation may exhibit undesirable properties (for example, undesirable Cₘₐₓ in patients upon administration and/or the inability to be manufactured on a commercial scale).

It will be understood that descriptions herein regarding the amount of omecamtiv mecarbil, a pharmaceutically acceptable salt thereof, or a pharmaceutically acceptable hydrate of a pharmaceutically acceptable salt thereof, is relative to the salt or hydrate form of the active ingredient. The amount of omecamtiv mecarbil described herein refers to the amount (or the equivalent amount) of omecamtiv mecarbil free base. For example, when a tablet formulation is indicated to have 1 mg of omecamtiv mecarbil, the tablet formulation comprises 1.22 mg of omecamtiv mecarbil dihydrochloride monohydrate (molecular weight (MW) of 492.37 g/mol) which provides 1 mg of omecamtiv mecarbil (MW of 401.43 g/mol).

In some embodiments, the tablet formulation comprises omecamtiv mecarbil, a pharmaceutical acceptable salt thereof, or a pharmaceutically acceptable hydrate of a pharmaceutically acceptable salt thereof, in an amount of 0.1 wt. % or more, based upon the total weight of the tablet formulation, for example, 0.5 wt. % or more, 1 wt. % or more, 2 wt. % or more, 3 wt. % or more, 4 wt. % or more, 5 wt. % or more, 6 wt. % or more, 7 wt. % or more, 8 wt. % or more, 9 wt. % or more, 10 wt. % or more, 11 wt. % or more, 12 wt. % or more, 13 wt. % or more, 14 wt. % or more, 15 wt. % or more, 16 wt. % or more, 17 wt. % or more, 18 wt. % or more, 19 wt. % or more, 20 wt. % or more, 21 wt. % or more, 22 wt. % or more, 23 wt. % or more, 24 wt. % or more, 25 wt. % or more, 26 wt. % or more, 27 wt. % or more, 28 wt. % or more, 29 wt. % or more, 30 wt. % or more, 31 wt. % or more, 32 wt. % or more, 33 wt. % or more, 34 wt. % or more, 35 wt. % or more, 36 wt. % or more, 37 wt. % or more, 38 wt. % or more, 39 wt. % or more, or 40 wt. % or more, of omecamtiv mecarbil, in whichever form (for example, salt, salt hydrate, or free base) the omecamtiv mecarbil is present in the tablet formulation. Alternatively, or in addition, the tablet formulation comprises omecamtiv mecarbil, a pharmaceutical acceptable salt thereof, or a pharmaceutically acceptable hydrate of a pharmaceutically acceptable salt thereof, in an amount of 80 wt. % or less, based upon the total weight of the tablet formulation, for example, 79 wt. % or less, 78 wt. % or less, 77 wt. % or less, 76 wt. % or less, 75 wt. % or less, 74 wt. % or less, 73 wt. % or less, 72 wt. % or less, 71 wt. % or less, 70 wt. % or less, 69 wt. % or less, 68 wt. % or less, 67 wt. % or less, 66 wt. % or less, 65 wt. % or less, 64 wt. % or less, 63 wt. % or less, 62 wt. % or less, 61 wt. % or less, 60 wt. % or less, 59 wt. % or less, 58 wt. % or less, 57 wt. % or less, 56 wt. % or less, 55 wt. % or less, 54 wt. % or less, 53 wt. % or less, 52 wt. % or less, 51 wt. % or less, 50 wt. % or less, 49 wt. % or less, 48 wt. % or less, 47 wt. % or less, 46 wt. % or less, 45 wt. % or less, 44 wt. % or less, 43 wt. % or less, 42 wt. % or less, or 41 wt. % or less, of omecamtiv mecarbil, in whichever form (for example, salt, salt hydrate, or free base) the omecamtiv mecarbil is present in the tablet formulation.

Thus, the tablet formulation comprises omecamtiv mecarbil, a pharmaceutical acceptable salt thereof, or a pharmaceutically acceptable hydrate of a pharmaceutically acceptable salt thereof, in an amount bounded by any two of the aforementioned endpoints. For example, the tablet formulation comprises omecamtiv mecarbil, a pharmaceutical acceptable salt thereof, or a pharmaceutically acceptable hydrate of a pharmaceutically acceptable salt thereof, in an amount of 0.1 to 80 wt. %, based upon the total weight of the tablet formulation, for example, 0.5 to 79 wt. %, 1 to 78 wt. %, 2 to 77 wt. %, 3 to 76 wt. %, 4 to 75 wt. %, 5 to 74 wt. %, 6 to 73 wt. %, 7 to 72 wt. %, 8 to 71 wt. %, 9 to 70 wt. %, 10 to 69 wt. %, 11 to 68 wt. %, 12 to 67 wt. %, 13 to 66 wt. %, 14 to 65 wt. %, 15 to 64 wt. %, 16 to 63 wt. %, 17 to 62 wt. %, 18 to 61 wt. %, 19 to 60 wt. %, 20 to 59 wt. %, 21 to 58 wt. %, 22 to 57 wt. %, 23 to 56 wt. %, 24 to 55 wt. %, 25 to 54 wt. %, 26 to 53 wt. %, 27 to 52 wt. %, 28 to 51 wt. %, 29 to 50 wt. %, 30 to 49 wt. %, 31 to 48 wt. %, 32 to 47 wt. %, 33 to 46 wt. %, 34 to 45 wt. %, 35 to 44 wt. %, 36 to 43 wt. %, 37 to 42 wt. %, 38 to 41 wt. %, or 39 to 40 wt. %, of omecamtiv mecarbil, in whichever form (for example, salt, salt hydrate, or free base) the omecamtiv mecarbil is present in the tablet formulation.

In some embodiments, the tablet formulation comprises omecamtiv mecarbil, a pharmaceutically acceptable salt thereof, or a pharmaceutically acceptable hydrate of a pharmaceutically acceptable salt thereof, in an amount of 5 to 10 wt. %, for example, 5.5 wt. %, 6 wt. %, 6.5 wt. %, 7 wt. %, 7.5 wt. %, 8 wt. %, 8.5 wt. %, 9 wt. %, or 9.5 wt. %, based upon the total weight of the tablet formulation.

In some embodiments, the core of the tablet formulation comprises omecamtiv mecarbil, a pharmaceutically acceptable salt thereof, or a pharmaceutically acceptable hydrate of a pharmaceutically acceptable salt thereof, in an amount of 8 wt. % (as the omecamtiv mecarbil free base), based on the total weight of the core.

In some embodiments, the tablet formulation comprises 1-3 mg omecamtiv mecarbil (free base), which can be present as omecamtiv mecarbil, a pharmaceutical acceptable salt thereof, or a pharmaceutically acceptable hydrate of a pharmaceutically acceptable salt thereof. In some embodiments, the tablet formulation comprises 1 mg of omecamtiv mecarbil (free base), which can be present as omecamtiv mecarbil, a pharmaceutical acceptable salt thereof, or a pharmaceutically acceptable hydrate of a pharmaceutically acceptable salt thereof.

### Tablet Cores

The tablet formulations disclosed herein comprise a core that is coated with a film coating. The core comprises omecamtiv mecarbil, a pharmaceutically acceptable salt thereof, or a pharmaceutically acceptable hydrate of a pharmaceutically acceptable salt thereof (as discussed above), a filler, a binder, a glidant, and a lubricant.

As described herein, the weight percentages of a particular component of a tablet formulation disclosed herein is based on the total weight of the tablet formulation (i.e., the entire tablet), unless otherwise specified. In some instances, it is more convenient to discuss the amount or concentration of a component based on the total weight of a portion of the tablet formulation (for example, the core or film coating).

The tablet formulation comprises one or more fillers. In some cases, the tablet formulation comprises one filler. In some cases, the tablet formulation comprises more than one filler (for example, two, three, or four fillers). The tablet formulation comprises any suitable amount of filler. If the tablet formulation comprises too little filler, the tablet formulation may exhibit undesirable properties, for example, an inability to be manufactured on a commercial scale. In contrast, if the tablet formulation comprises too much filler, the tablet formulation may exhibit undesirable properties (for example, increasing the pill burden to the patient).

As used herein, the term “filler” refers a substance that can be added to components of a pharmaceutical formulation to increase bulk weight of the material to be formulated, for example tableted, in order to achieve the desired weight. Fillers include but are not limited to starches, lactose, cellulose derivatives, sugar alcohols and the like. Different grades of starches include, but are not limited to, maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different grades of lactose include, but are not limited, to lactose monohydrate, lactose DT (direct tableting), lactose anhydrous, Flowlac™ (available from Meggle products), Pharmatose™ (available from DMV) and others. Different cellulose derivatives that can be used include crystalline cellulose, such as microcrystalline cellulose, and powdered cellulose. Different sugar alcohols that can be used include mannitol (such as Pearlitol™ SD 200), sorbitol, and xylitol

In some cases, the filler comprises microcrystalline cellulose having a particle size of 50 μm and moisture content of 3 to 5% (for example, Avicel PH101), or microcrystalline cellulose having a particle size of 100 μm and moisture content of 3 to 5% (for example, Avicel PH102), or microcrystalline cellulose having a particle size of 180 μm and moisture content of 2 to 5% (for example, Avicel PH200) or lactose monohydrate, or crystalline 325 mesh impalpable lactose monohydrate (for example, Unisweet L-313 or Pharmatose 110M) or crystalline 200 mesh, impalpable lactose monohydrate (for example, Unisweet L-312), or a spray-dried mixture of crystalline and amorphous lactose monohydrate having an particle size of 60-120 μm (for example, Fast Flo 316), or a combination thereof.

The tablet formulation comprises 20 wt. % or more filler, based on the total weight of the tablet formulation, for example, 21 wt. % or more, 22 wt. % or more, 23 wt. % or more, 24 wt. % or more, 25 wt. % or more, 26 wt. % or more, 27 wt. % or more, 28 wt. % or more, 29 wt. % or more, 30 wt. % or more, 31 wt. % or more, 32 wt. % or more, 33 wt. % or more, 34 wt. % or more, 35 wt. % or more, 36 wt. % or more, 37 wt. % or more, 38 wt. % or more, 39 wt. % or more, 40 wt. % or more, 41 wt. % or more, 42 wt. % or more, 43 wt. % or more, 44 wt. % or more, 45 wt. % or more, 46 wt. % or more, 47 wt. % or more, 48 wt. % or more, 49 wt. % or more, or 50 wt. % or more filler, based on the total weight of the tablet formulation. Alternatively, or in addition, the tablet formulation comprises 90 wt. % or less filler, based on the total weight of the tablet formulation, for example, 89 wt. % or less, 88 wt. % or less, 87 wt. % or less, 86 wt. % or less, 85 wt. % or less, 84 wt. % or less, 83 wt. % or less, 82 wt. % or less, 81 wt. % or less, 80 wt. % or less, 79 wt. % or less, 78 wt. % or less, 77 wt. % or less, 76 wt. % or less, 75 wt. % or less, 74 wt. % or less, 73 wt. % or less, 72 wt. % or less, 71 wt. % or less, 70 wt. % or less, 69 wt. % or less, 68 wt. % or less, 67 wt. % or less, 66 wt. % or less, 65 wt. % or less, 64 wt. % or less, 63 wt. % or less, 62 wt. % or less, 61 wt. % or less, 60 wt. % or less, 59 wt. % or less, 58 wt. % or less, 57 wt. % or less, 56 wt. % or less, 55 wt. % or less, 54 wt. % or less, 53 wt. % or less, 52 wt. % or less, or 51 wt. % or less filler, based on the total weight of the tablet formulation.

Thus, the tablet formulation comprises a filler in an amount bounded by any two of the aforementioned endpoints. For example, the tablet formulation comprises 20-90 wt. % filler, based on the total weight of the tablet formulation, for example, 21-89 wt. %, 22-88 wt. %, 23-87 wt. %, 24-86 wt. %, 25-85 wt. %, 26-84 wt. %, 27-83 wt. %, 28-82 wt. %, 29-81 wt. %, 30-80 wt. %, 31-79 wt. %, 32-78 wt. %, 33-77 wt. %, 34-76 wt. %, 35-75 wt. %, 36-74 wt. %, 37-73 wt. %, 38-72 wt. %, 39-71 wt. %, 40-70 wt. %, 41-69 wt. %, 42-68 wt. %, 43-67 wt. %, 44-66 wt. %, 45-65 wt. %, 46-64 wt. %, 47-63 wt. %, 48-62 wt. %, 49-61 wt. %, 50-60 wt. %, 51-59 wt. %, 52-58 wt. %, 53-57 wt. %, or 54-56 wt. % filler, based on the total weight of the tablet formulation.

In some embodiments, the tablet formulation comprises 60 wt. % to 90 wt. %, 77.9 wt. %, 74.5%, or 65.9% of filler, based on the total weight of the tablet formulation. In some embodiments, the core of the tablet formulation comprises 85.6 wt. % of filler, based on the total weight of the core of the tablet formulation.

In some embodiments, the filler comprises microcrystalline cellulose, lactose monohydrate, or a combination thereof. More than one filler (for example, 2, 3, 4 or more fillers) can be present in a tablet formulation as disclosed herein. For example, in some embodiments, the filler comprises microcrystalline cellulose and lactose monohydrate. In embodiments wherein the tablet formulation comprises more than one filler, it is understood that the total amount of filler present falls within the amounts described herein.

In some embodiments, the tablet formulation comprises 10-45 wt. % microcrystalline cellulose and 10-45 wt. % lactose monohydrate, based on the total weight of the tablet formulation, for example, 10-40 wt. % microcrystalline cellulose and 10-40 wt. % lactose monohydrate, 30-40 wt. % microcrystalline cellulose and 30-40 wt. % lactose monohydrate, 39 wt. % microcrystalline cellulose and 39 wt. % lactose monohydrate, 38 wt. % microcrystalline cellulose and 38 wt. % lactose monohydrate, 37 wt. % microcrystalline cellulose and 37 wt. % lactose monohydrate, 36 wt. % microcrystalline cellulose and 36 wt. % lactose monohydrate, 35 wt. % microcrystalline cellulose and 35 wt. % lactose monohydrate, 34 wt. % microcrystalline cellulose and 34 wt. % lactose monohydrate, or 33 wt. % microcrystalline cellulose and 33 wt. % lactose monohydrate, 32 wt. % microcrystalline cellulose and 32 wt. % lactose monohydrate, or 31 wt. % microcrystalline cellulose and 31 wt. % lactose monohydrate as filler, based on the total weight of the tablet formulation. In some embodiments, the core of the tablet formulation comprises 42.8 wt. % of microcrystalline cellulose and 42.8 wt. % of lactose monohydrate, based on the total weight of the core of the tablet formulation.

In some embodiments, the tablet formulations disclosed herein comprise a binder. In some cases, the tablet formulation comprises one binder. In some cases, the tablet formulation comprises more than one binder (for example, two, three, or four binder). The tablet formulation comprises any suitable amount of binder. If the tablet formulation comprises too little binder the tablet formulation may, for example, lack stability. In contrast, if the tablet formulation comprises too much binder, the tablet formulation may exhibit undesirable pharmacokinetic properties (for example, slow release rate).

As used herein, the term “binder” refers to a substance used in the tablet formulation to hold the active pharmaceutical ingredient and inactive ingredients together in a cohesive granule. Suitable binders include but are not limited to, for example, carboxymethycellulose sodium USP, hypromellose USP, hydroxyethyl cellulose NF, and hydroxypropyl cellulose NF. In addition, other binders include polyvidone, polyvinyl pyrrolidone, gelatin NF, natural gums (such as acacia, tragacanth, guar, and pectin), starch paste, pregelatinized starch NF, sucrose NF, corn syrup, polyethylene glycols, and sodium alginate, ammonium calcium alginate, magnesium aluminum silicate, polyethylene glycols. In an embodiment, the binder comprises hydroxypropyl cellulose.

In some embodiments, the binder comprises a hydroxypropyl cellulose (HPC) having a Brookfield viscosity of 300-600 mPa-s (10%) (for example, Klucel EXF).

In various embodiments, the tablet formulation comprises 0.5 wt. % or more binder, based on the total weight of the tablet formulation, for example, 1 wt. % or more, 1.5 wt. % or more, 2 wt. % or more, 2.5 wt. % or more, 3 wt. % or more, 3.5 wt. % or more, 4 wt. % or more, 4.5 wt. % or more, 5 wt. % or more, 5.5 wt. % or more, 6 wt. % or more, 6.5 wt. % or more, 7 wt. % or more, or 7.5 wt. % or more binder, based on the total weight of the tablet formulation. Alternatively, or in addition, the tablet formulation comprises 15 wt. % or less binder, for example, 14.5 wt. % or less, 14 wt. % or less, 13.5 wt. % or less, 13 wt. % or less, 12.5 wt. % or less, 12 wt. % or less, 11.5 wt. % or less, 11 wt. % or less, 10.5 wt. % or less, 10 wt. % or less, 9.5 wt. % or less, 9 wt. % or less, 8.5 wt. % or less, or 8 wt. % or less binder, based on the total weight of the tablet formulation.

Thus, the tablet formulation comprises binder in an amount bounded by any of the aforementioned endpoints. For example, the tablet formulation comprises 0.5-15 wt. % binder, for example, 1-14.5 wt. %, 1.5-14 wt. %, 2-13.5 wt. %, 2.5-13 wt. %, 3-12.5 wt. %, 3.5-12 wt. %, 4-11.5 wt. %, 4.5-11 wt. %, 5-10.5 wt. %, 5.5-10 wt. %, 6-9.5 wt. %, 6.5-9 wt. %, 7-8.5 wt. %, or 7.5-8 wt. % binder, based on the total weight of the tablet formulation. In some embodiments, the tablet formulation comprises 1-8 wt. % binder, for example, 2-5 wt. %, or 2-4 wt. % binder. In some embodiments, the core of the tablet formulation comprises 2-5 wt. % binder, based on the total weight of the tablet formulation. In any of the aforementioned embodiments, the binder may be HPC.

In embodiments where the binder, for example, comprises HPC, the tablet formulation comprises 1-8 wt. % HPC, based on the total weight of the tablet formulation, for example, 1-5 wt. % HPC, based on the total weight of the tablet formulation. In some embodiments, the core of the tablet formulation comprises 3 wt. % HPC, based on the total weight of the core of the tablet formulation. In some embodiments, the tablet formulation comprises 2.7 wt. % HPC, based on the total weight of the tablet formulation. In some embodiments, the tablet formulation comprises 2.6 wt. % HPC, based on the total weight of the tablet formulation. In various embodiments, the tablet formulation comprises 2.3 wt. % HPC, based on the total weight of the tablet formulation. In some embodiments, the core of the tablet formulation comprises 3 wt. % of HPC, based on the total weight of the core of the tablet formulation.

In some embodiments, the tablet formulations disclosed herein comprise a glidant. In some cases, the tablet formulation comprises one glidant. In some cases, the tablet formulation comprises more than one glidant (for example, two, three, or four glidants). The glidant is an extra-granular component added to improve flowability of the composition. The tablet formulation comprises any suitable amount of glidant. If the composition comprises too little glidant, the tablet formulation can exhibit poor flowability and be difficult to process during manufacture. In contrast, if the composition comprises too much glidant, the tablet formulation may exhibit undesirable handling properties and/or not be cost effective.

As used herein, the term “glidant” refers to a substance that is added to a powder formulation blend to improve its flowability. Suitable glidants include but are not limited to, for example, silica, colloidal silicon dioxide, colloidal silica anhydrous (for example, Aerosil 200), magnesium trisilicate, powdered cellulose, starch, talc, and combinations thereof.

The tablet formulations disclosed herein comprise 0.1 wt. % or more glidant, based on the total weight of the tablet formulation, for example, 0.25 wt. % or more, 0.5 wt. % or more, 0.75 wt. % or more, 1 wt. % or more, 1.25 wt. % or more, 1.5 wt. % or more, 1.75 wt. % or more, 2 wt. % or more, 2.25 wt. % or more, 2.5 wt. % or more, 2.75 wt. % or more, or 3 wt. % or more glidant, based on the total weight of the tablet formulation. Alternatively, or in addition the tablet formulation comprises 5 wt. % or less glidant, for example, 4.75 wt. % or less, 4.5 wt. % or less, 4.25 wt. % or less, 4 wt. % or less, 3.75 wt. % or less, 3.5 wt. % or less, or 3.25 wt. % or less glidant, based on the total weight of the tablet formulation.

Thus, the tablet formulations comprise a glidant in an amount bounded by any of the aforementioned endpoints. For example, the tablet formulations comprise 0.1-5 wt. % glidant, based on the total weight of the tablet formulation, for example, 0.25-4.75 wt. %, 0.5-4.5 wt. %, 0.75-4.25 wt. %, 1-4 wt. %, 1.25-3.75 wt. %, 1.5-3.5 wt. %, 1.75-3.25 wt. %, 2-3 wt. %, or 2.25-2.75 wt. % glidant, based on the total weight of the tablet formulation. In some embodiments, the tablet formulation comprises 0.1-5 wt. % glidant, for example, 0.1-2 wt. % glidant or 0.2-1 wt. % glidant, based on the total weight of the tablet formulation. In some embodiments, the core of the tablet formulation comprises 0.2-0.8 wt. % glidant, based on the total weight of the tablet formulation. In any of the aforementioned embodiments, the glidant may be colloidal silicon dioxide.

In embodiments where the glidant, for example, comprises colloidal silicon dioxide, the tablet formulation comprises 0.5 wt. % colloidal silicon dioxide, based on the total weight of the tablet formulation. In some embodiments, the tablet formulation comprises 0.4 wt. % colloidal silicon dioxide, based on the total weight of the tablet formulation. In some embodiments, the core of the tablet formulation comprises 0.5 wt. % colloidal silicon dioxide, based on the total weight of the core of the tablet formulation.

In some embodiments, the tablet formulations disclosed herein comprise a lubricant. In some cases, the tablet formulation comprises one lubricant. In some cases, the tablet formulation comprises more than one lubricant (for example, two, three, or four lubricants). The lubricant is an extra-granular component added to improve the handling of the composition. The tablet formulation comprises any suitable amount of lubricant. If the composition comprises too little lubricant, the tablet formulation can exhibit poor handling properties and be difficult to process during manufacture. In contrast, if the composition comprises too much lubricant, the tablet formulation may exhibit undesirable properties and/or not be cost effective.

As used herein, the term “lubricant” refers to a substance that can be added to components of the present tablet formulations to reduce sticking by a solid formulation to the equipment used for the production of a unit dosage form. Lubricants include stearic acid, hydrogenated vegetable oils, hydrogenated soybean oil and hydrogenated soybean oil & castor wax, stearyl alcohol, leucine, magnesium stearate, glycerylmonostearate, stearic acid, glycerybehenate, ethylene oxide polymers, sodium lauryl sulfate, magnesium lauryl sulfate, sodium oleate, sodium stearyl fumarate, and dl-leucine, and mixtures thereof. In some cases, the lubricant comprises magnesium stearate.

The tablet formulations disclosed herein comprise 0.2 wt. % or more lubricant, based on the total weight of the tablet formulation, for example, 0.25 wt. % or more, 0.3 wt. % or more, 0.4 wt. % or more, 0.5 wt. % or more, 0.6 wt. % or more, 0.7 wt. % or more, 0.8 wt. % or more, 0.9 wt. % or more, or 1 wt. % or more lubricant, based on the total weight of the tablet formulation. Alternatively, or in addition the tablet formulation comprises 2 wt. % or less lubricant, based on the total weight of the tablet formulation, for example, 1.9 wt. % or less, 1.8 wt. % or less, 1.7 wt. % or less, 1.6 wt. % or less, 1.5 wt. % or less, 1.4 wt. % or less, 1.3 wt. % or less, 1.2 wt. % or less, or 1.1 wt. % or less lubricant, based on the total weight of the tablet formulation.

Thus, a tablet formulation disclosed herein comprises a lubricant in an amount bounded by any of the aforementioned endpoints. For example, the tablet formulation comprises 0.2-2 wt. % lubricant, based on the total weight of the tablet formulation, for example, 0.25-1.9 wt. %, 0.3-1.8 wt. %, 0.4-1.7 wt. %, 0.5-1.6 wt. %, 0.6-1.5 wt. %, 0.7-1.4 wt. %, 0.8-1.3 wt. %, 0.9-1.2 wt. %, or 1-1.1 wt. % lubricant, based on the total weight of the tablet formulation. In some embodiments, the core of the tablet formulation comprises 0.8-1.2 wt. % lubricant (for example, 1 wt. % lubricant), based on the total weight of the tablet formulation. In any of the aforementioned embodiments, the lubricant may be magnesium stearate.

In some embodiments, wherein the lubricant, for example, comprises magnesium stearate, the tablet formulation comprises 0.25-3 wt. % magnesium stearate, based on the total weight of the tablet formulation. In various embodiments, wherein the lubricant comprises magnesium stearate, the tablet formulation comprises 0.5-3 wt. % magnesium stearate, based on the total weight of the tablet formulation. In some embodiments, the tablet formulation comprises 1 wt. % magnesium stearate, based on the total weight of the tablet formulation. In various embodiments, the tablet formulation comprises 0.9 wt. % magnesium stearate, based on the total weight of the tablet formulation. In some embodiments, the tablet formulation comprises 0.8 wt. % magnesium stearate, based on the total weight of the tablet formulation. In some embodiments, the core of the tablet formulation comprises 1.0 wt. % magnesium stearate, based on the total weight of the core of the tablet formulation.

In various embodiments, the tablet formulations disclosed herein do not contain, or are substantially free of, a pH-modifier. Examples of pH modifiers include maleic acid, citric acid, malic acid, fumaric acid, sulfuric acid, tartaric acid, lactic acid, salicylic acid, aspartic acid, aminosalicylic acid, malonic acid, glutamic acid, or a combination thereof. In some cases, a pH modifier is fumaric acid.

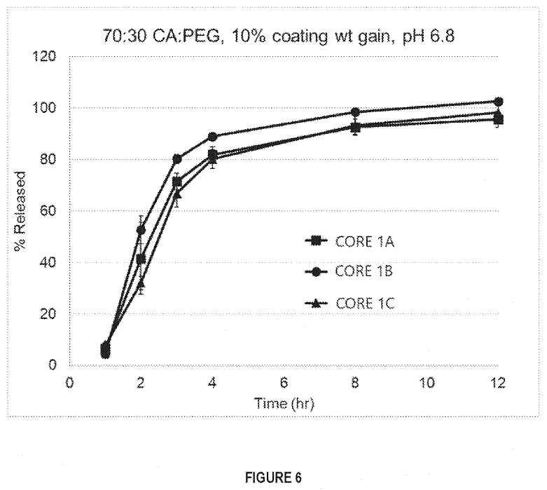

Applicant surprisingly discovered that cores containing fumaric acid do not exhibit an enhanced release of omecamtiv mecarbil in 6.8 buffer. Without wishing to be bound to any particular theory, it is believed that fumaric acid acts as a pH modifier by providing a low microenvironmental pH inside an immediate release mini-tablet core at neutral pH 6.8 environment. The low pH inside the mini-tablet core helps to solubilize omecamtiv mecarbil, which in turn enhances omecamtiv mecarbil release at neutral pH 6.8. However, as shown herein with the data in FIGS. 6-8, fumaric acid did not enhance omecamtiv mecarbil release in 6.8 buffer. Without wishing to be bound to any particular theory, it is believed that this effect may be due to neutralization of the fumaric acid by buffer ions at the beginning of dissolution resulting in the loss of ability to provide low pH inside mini-tablet in pH 6.8 buffer. The data in FIGS. 6-8 surprisingly show that fumaric acid did not enhance omecamtiv mecarbil release at neutral pH, and therefore suggest that fumaric acid is not a required component for the immediate-release mini-tablet formulation.

In some embodiments, the core of the tablet formulation comprises 8-11 wt. % of omecamtiv mecarbil, a pharmaceutically acceptable salt thereof, or a pharmaceutically acceptable hydrate of a pharmaceutically acceptable salt thereof; 83-86 wt. % filler; 2-5 wt. % binder; 0.2-0.8 wt. % glidant; and 0.8-1.2 wt. % lubricant, based upon the total weight of the core.

### Film Coating

The tablet formulations disclosed herein comprise a film coating on the core. The film coating comprises a modified-release polymer (i.e., a control release agent) and a pore former. In some embodiments, the film coating further comprises a plasticizer. In some embodiments, the pore former also acts as a plasticizer.

As used herein, the term “modified-release polymer” (sometimes alternatively referred to herein as a “control release agent”) refers to a substance that facilitates the release of omecamtiv mecarbil, a pharmaceutically acceptable salt thereof, or a pharmaceutically acceptable hydrate of a pharmaceutically acceptable salt thereof, from a tablet formulation in a controlled fashion. In some cases, the film coating comprises one modified-release polymer. In some cases, the film coating comprises more than one modified-release polymer (for example, two, three, or four modified-release polymers). Modified-release polymers can form a semi-permeable film upon hydration. Examples of modified-release polymers include but are not limited to, cellulose acetate (CA), copolymer of ethyl acrylate and methyl methacrylate (for example, poly(ethyl acrylate-co-methyl methacrylate-co-trimethylammonioethyl methacrylate chloride 1:2:0.1 (Eudragit RS); and poly(ethyl acrylate-co-methyl methacrylate-co-trimethylammonioethyl methacrylate chloride 1:2:0.2 (Eudragit RL), ethyl cellulose, or polyvinyl acetate, or a combination thereof.

In various embodiments, the modified-release polymer of the film coating comprises ethylcellulose, poly(ethyl acrylate-co-methylmethacrylate), poly(ethyl acrylate-co-methylmethacrylate-co-trimethylammonioethylmethacrylate chloride), cellulose acetate, polyvinyl acetate, or a combination thereof. In some embodiments, the modified-release polymer comprises cellulose acetate.

The tablet formulation comprises any suitable amount of modified-release polymer. If the tablet formulation comprises too little modified-release polymer, the tablet formulation may exhibit a rapid release rate of omecamtiv mecarbil. In contrast, if the tablet formulation comprises too much control release agent, the tablet formulation may exhibit a release rate of omecamtiv mecarbil that is too slow.

In embodiments wherein the modified-release polymer comprises, for example, cellulose acetate, the tablet formulation comprises 3 wt. % or more cellulose acetate of the total weight of the tablet formulation, for example, 4 wt. % or more, 5 wt. % or more, 6 wt. % or more, 7 wt. % or more, 8 wt. % or more, 9 wt. % or more, 10 wt. % or more, 11 wt. % or more, or 12 wt. % or more cellulose acetate, based upon the total weight of the tablet formulation. Alternatively, or in addition, the tablet formulation comprises 20 wt. % or less cellulose acetate of the total weight of the tablet formulation, for example, 19 wt. % or less, 18 wt. % or less, 17 wt. % or less, 16 wt. % or less, 15 wt. % or less, 14 wt. % or less, or 13 wt. % or less cellulose acetate, based upon the total weight of the tablet formulation.

Thus, the tablet formulation comprises cellulose acetate in any amount bounded by the aforementioned endpoints. For example, the tablet formulation comprises 3-20 wt. % cellulose acetate of the total weight of the tablet formulation, for example, 4-19 wt. %, 5-18 wt. %, 6-17 wt. %, 7-16 wt. %, 8-15 wt. %, 9-14 wt. %, 10-13 wt. %, or 11-12 wt. % cellulose acetate, based upon the total weight of the tablet formulation. In some embodiments, the tablet formulation comprises 5.4 wt. % cellulose acetate, based upon the total weight of the tablet formulation. In some embodiments, the tablet formulation comprises 7.8 wt. % cellulose acetate, based upon the total weight of the tablet formulation. In some embodiments, the tablet formulation comprises 13.8 wt. % cellulose acetate, based upon the total weight of the tablet formulation.

In some embodiments, the film coating comprises 40 wt. % or more modified-release polymer, based upon the total weight of the film coating, for example, 41 wt. % or more, 42 wt. % or more, 43 wt. % or more, 44 wt. % or more, 45 wt. % or more, 46 wt. % or more, 47 wt. % or more, 48 wt. % or more, 49 wt. % or more, 50 wt. % or more, 51 wt. % or more, 52 wt. % or more, 53 wt. % or more, 54 wt. % or more, 55 wt. % or more, 56 wt. % or more, 57 wt. % or more, 58 wt. % or more, 59 wt. % or more, 60 wt. % or more, 61 wt. % or more, 62 wt. % or more, 63 wt. % or more, 64 wt. % or more, or 65 wt. % or more modified-release polymer, based upon the total weight of the film coating. Alternatively, or in addition, the film coating comprises 90 wt. % or less modified-release polymer of the total weight of the film coating, for example, 89 wt. % or less, 88 wt. % or less, 87 wt. % or less, 86 wt. % or less, 85 wt. % or less, 84 wt. % or less, 83 wt. % or less, 82 wt. % or less, 81 wt. % or less, 80 wt. % or less, 79 wt. % or less, 78 wt. % or less, 77 wt. % or less, 76 wt. % or less, 75 wt. % or less, 74 wt. % or less, 73 wt. % or less, 72 wt. % or less, 71 wt. % or less, 70 wt. % or less, 69 wt. % or less, 68 wt. % or less, 67 wt. % or less, or 66 wt. % or less modified-release polymer, based upon the total weight of the film coating.

Thus, the film coating comprises an amount of modified-release polymer in any amount bounded by two of the aforementioned endpoints. For example, the film-coating comprises 40-90 wt. % modified-release polymer of the total weight of the film coating, based upon the total weight of the film coating, for example, 41-89 wt. %, 42-88 wt. %, 43-87 wt. %, 44-86 wt. %, 45-85 wt. %, 46-84 wt. %, 47-83 wt. %, 48-82 wt. %, 49-81 wt. %, 50-80 wt. %, 51-79 wt. %, 52-78 wt. %, 53-77 wt. %, 54-76 wt. %, 55-75 wt. %, 56-74 wt. %, 57-73 wt. %, 58-72 wt. %, 59-71 wt. %, 60-70 wt. %, 61-69 wt. %, 62-68 wt. %, 63-67 wt. %, or 64-66 wt. % modified-release polymer, based upon the total weight of the film coating.

In some embodiments, the film coating comprises 40-90 wt. % modified-release polymer, 50-80 wt. %, 60-70 wt. %, or 55-65 wt. % modified-release polymer, based upon the total weight of the film coating. In some embodiments, the film coating comprises 60 wt. % modified release polymer, based upon the total weight of the film coating.

As used herein, the term “pore former” refers to a substance that increases the porosity of a water insoluble film and facilitates drug diffusion. Upon exposure to water or a biological fluid, the pore former dissolves and forms drug release channels in a water insoluble film barrier. In some cases, the film coating comprises one pore former. In some cases, the film coating comprises more than one pore former (for example, two, three, or four pore formers). Suitable pore formers of the film coating include but are not limited to, hydroxypropyl methylcellulose, polyvinylpyrrolidone, sorbitol, triethyl citrate, polyethylene glycol, or a combination thereof. Suitable pore formers include but are not limited to, for example, polyethylene glycols (for example, PEG 3350), sorbitol, hypromellose having methoxy content of 28-30% and a hydroxypropyl content of 7-12% and having a viscosity of 4-6 cP at 20° C., 2% in water (for example, Methocel E5), hypromellose having methoxy content of 28-30% and a hydroxypropyl content of 7-12% and having a viscosity of 5-7 cP at 20° C., 2% in water (for example, Methocel E6), or polyvinyl alcohol-polyethylnene glycol graft copolymer having a molecular weight of 45 kDa, a melting point of 209° C., and a viscosity of 115 mPa-s as a 20% solution (for example, Kollicoat IR), or a combination thereof.

In some embodiments, the pore former comprises polyethylene glycol (PEG). An exemplary polyethylene glycol is PEG 3350 (CAS No. 25322-68-3) having a molecular weight (MW) of 3350 g/mol and a melting point of 56° C.

As used herein, the term “plasticizer” refers to a substance that decreases the plasticity or decreases the attraction between polymer chains to make them more flexible to prevent polymer film crack formation or peeling off. In some cases, the film coating comprises one plasticizer. In some cases, the film coating comprises more than one plasticizer (for example, two, three, or four plasticizers). Suitable plasticizers include but are not limited to, for example, polyethylene glycols (for example, PEG 3350), diethyl phthalate, triethyl citrate, dibutyl sebacate, or triacetin, or a combination thereof. In some cases, the pore former can also exhibit plasticizer properties and a component can be both pore former and plasticizer. Suitable plasticizers include but are not limited to, PEG, diethyl phthalate, triethyl citrate, dibutyl sebacate, triacetin, or a combination thereof. In some cases, the plasticizer comprises PEG.

In some embodiments, the pore former is also a plasticizer. In some cases, the pore former, which is also a plasticizer, comprises PEG, for example, PEG 3350.

The tablet formulation comprises any suitable amount of pore former. If the tablet formulation comprises too little pore former, the tablet formulation may exhibit an omecamtiv mecarbil release rate that is too slow. In contrast, if the tablet formulation comprises too much pore former, the tablet formulation may exhibit an omecamtiv mecarbil release rate that is too fast.

The tablet formulation comprises 2 wt. % or more pore former based upon total weight of the tablet formulation, for example, 3 wt. % or more, 4 wt. % or more, 5 wt. % or more, 6 wt. % or more, 7 wt. % or more, 8 wt. % or more, 9 wt. % or more, 10 wt. % or more, 11 wt. % or more, 12 wt. % or more, 13 wt. % or more, 14 wt. % or more, 15 wt. % or more, 16 wt. % or more, 17 wt. % or more, 18 wt. % or more, 19 wt. % or more, 20 wt. % or more, 21 wt. % or more, 22 wt. % or more, 23 wt. % or more, 24 wt. % or more, 25 wt. % or more, 26 wt. % or more, 27 wt. % or more, or 28 wt. % or more pore former, based upon the total weight of the tablet formulation. Alternatively, or in addition, the tablet formulation comprises 50 wt. % or less pore former based upon total weight of the tablet formulation, for example, 49 wt. % or less, 48 wt. % or less, 47 wt. % or less, 46 wt. % or less, 45 wt. % or less, 44 wt. % or less, 43 wt. % or less, 42 wt. % or less, 41 wt. % or less, 40 wt. % or less, 39 wt. % or less, 38 wt. % or less, 37 wt. % or less, 36 wt. % or less, 35 wt. % or less, 34 wt. % or less, 33 wt. % or less, 32 wt. % or less, 31 wt. % or less, 30 wt. % or less, or 29 wt. % or less pore former, based upon the total weight of the tablet formulation.

Thus, the tablet formulation comprises pore former in an amount bounded by any two of the aforementioned endpoints. For example, the film coating comprises 2-50 wt. % pore former based upon total weight of the tablet formulation, for example, 3-49 wt. %, 4-48 wt. %, 5-47 wt. %, 6-46 wt. %, 7-45 wt. %, 8-44 wt. %, 9-43 wt. %, 10-42 wt. %, 11-41 wt. %, 12-40 wt. %, 13-39 wt. %, 14-38 wt. %, 15-37 wt. %, 16-36 wt. %, 17-35 wt. %, 18-34 wt. %, 19-33 wt. %, 20-32 wt. %, 21-31 wt. %, 22-30 wt. %, 23-29 wt. %, 24-28 wt. %, or 25-27 wt. % pore former, based upon the total weight of the tablet formulation. In some embodiments, the film coating comprises 2-50 wt. % pore former, or 20-40 wt. % pore former, based upon the total weight of the tablet formulation.

In embodiments where the pore former comprises, for example, PEG, the tablet formulation comprises 2-15 wt. % PEG based upon total weight of the tablet formulation, for example, 3, 4, 5, 6, 7, 8, 9, 10, 11, 12, 13, or 14 wt. % PEG, based upon the total weight of the tablet formulation. In some embodiments, the tablet formulation comprises 3.6 wt. % PEG based upon total weight of the tablet formulation. In various embodiments, the tablet formulation comprises 5.2 wt. % PEG based upon total weight of the tablet formulation. In still various embodiments, the tablet formulation comprises 9.2 wt. % PEG based upon total weight of the tablet formulation.

The tablet formulation comprises any suitable amount of plasticizer. If the tablet formulation comprises too little plasticizer, the film coating may be brittle and easily broken. In contrast, if the tablet formulation comprises too much plasticizer, the film coating may exhibit a high level of tackiness and a less robust coating process.

The tablet formulation comprises 0.25 wt. % or more plasticizer based upon total weight of the tablet formulation, for example, 0.3 wt. % or more, 0.4 wt. % or more, 0.5 wt. % or more, 0.6 wt. % or more, 0.7 wt. % or more, 0.8 wt. % or more, 0.9 wt. % or more, 1 wt. % or more, 1.1 wt. % or more, 1.2 wt. % or more, 1.3 wt. % or more, 1.4 wt. % or more, 1.5 wt. % or more, 1.6 wt. % or more. 1.7 wt. % or more, 1.8 wt. % or more, 1.9 wt. % or more, 2 wt. % or more, 2.1 wt. % or more, 2.2 wt. % or more, 2.3 wt. % or more, 2.4 wt. % or more, or 2.5 wt. % or more plasticizer, based upon the total weight of the tablet formulation. Alternatively, or in addition, the tablet formulation comprises 5 wt. % or less based upon total weight of the tablet formulation, for example, 4.9 wt. % or less, 4.8 wt. % or less, 4.7 wt. % or less, 4.6 wt. % or less, 4.5 wt. % or less, 4.4 wt. % or less, 4.3 wt. % or less, 4.2 wt. % or less, 4.1 wt. % or less, 4.0 wt. % or less, 3.9 wt. % or less, 3.8 wt. % or less, 3.7 wt. % or less, 3.6 wt. % or less, 3.5 wt. % or less, 3.4 wt. % or less, 3.3 wt. % or less, 3.2 wt. % or less, 3.1 wt. % or less, 3 wt. % or less, 2.9 wt. % or less, 2.8 wt. % or less, 2.7 wt. % or less, or 2.6 wt. % or less plasticizer, based upon the total weight of the tablet formulation.

Thus, the tablet formulation comprises plasticizer in an amount bounded by any two of the aforementioned endpoints. For example, the tablet formulation comprises 0.25-5.0 wt. % plasticizer based upon total weight of the tablet formulation, for example, 0.3-4.9 wt. %, 0.4-4.8 wt. %, 0.5-4.7 wt. %, 0.6-4.6 wt. %, 0.7-4.5 wt. %, 0.8-4.4 wt. %, 0.9-4.3 wt. %, 1.0-4.2 wt. %, 1.1-4.1 wt. %, 1.2-4.0 wt. %, 1.3-3.9 wt. %, 1.4-3.8 wt. %, 1.5-3.7 wt. %, 1.6-3.6 wt. %, 1.7-3.5 wt. %, 1.8-3.4 wt. %, 1.9-3.2 wt. %, 2.0-3.1 wt. %, 2.1-3.0 wt. %, 2.2-2.9 wt. %, 2.3-2.8 wt. %, 2.4-2.7 wt. %, or 2.5-2.6 wt. % plasticizer, based upon the total weight of the tablet formulation.

In some embodiments, the tablet formulation comprises 0.25-5.0 wt. % plasticizer based upon total weight of the tablet formulation.

The film coating, as well as each component of the film coating, is present in an amount to provide a desirable release profile of omecamtiv mecarbil. Accordingly, the amount of the film coating, as well as the composition of the film coating, can be adjusted to modulate the release of omecamtiv mecarbil.

In some embodiments, the film coating of the tablet formulation comprises 50-90 wt. % modified-release polymer and 10-50 wt. % pore former and plasticizer (when present), based upon total weight of the film coating, for example 60 wt. % modified-release polymer and 40 wt. % pore former and plasticizer (when present), based upon the total weight of the tablet formulation. In some embodiments, the film coating comprises 60 wt. % modified-release polymer and 40 wt. % pore former and plasticizer (when present), based upon total weight of the film coating. In some embodiments, the film coating comprises 9 wt. % of the total weight of the tablet formulation. In some embodiments, the film coating comprises 13 wt. % of the total weight of the tablet formulation. In some embodiments, the film coating comprises 23 wt. % of the total weight of the tablet formulation. The coating can be applied to the core using any suitable coating method. Nonlimiting coating methods include, for example, pan coating and fluid bed coating methods.

In some cases, the coating is applied using a coating composition. The coating composition comprises a coating solvent, the modified-release polymer, pore former, and plasticizer (when present). Any suitable coating solvent(s) can be used to prepare the coating composition. Suitable coating solvents include but are not limited water, acetone, and any combination thereof. The coating composition can have 5-10 wt. % solids and 90-95 wt. % solvent.

In some embodiments, the coating solvent comprises acetone and water (for example, 9:1 by weight).

### Tablet Formulations

In some embodiments, the tablet formulation comprises 5-40 wt. % omecamtiv mecarbil dihydrochloride monohydrate; 10-45 wt. % microcrystalline cellulose; 10-45 wt. % lactose monohydrate; 1-8 wt. % hydroxypropyl cellulose; 0.1-2 wt. % colloidal silicon dioxide; 0.25-3 wt. % magnesium stearate; 3-20 wt. % cellulose acetate; and 2-15 wt. % polyethylene glycol, based upon the total weight of the tablet formulation.

In some embodiments, the tablet formulation comprises 5-10 wt. % omecamtiv mecarbil dihydrochloride monohydrate; 30-45 wt. % microcrystalline cellulose; 30-45 wt. % lactose monohydrate; 1-5 wt. % hydroxypropyl cellulose; 0.1-2 wt. % colloidal silicon dioxide; 0.5-3 wt. % magnesium stearate; 3-20 wt. % cellulose acetate; and 2-15 wt. % polyethylene glycol, based upon the total weight of the tablet formulation.

In some embodiments, the tablet formulation comprises 9 wt. % omecamtiv mecarbil dihydrochloride monohydrate; 38.9 wt. % microcrystalline cellulose; 38.9 wt. % lactose monohydrate; 2.7 wt. % hydroxypropyl cellulose; 0.5 wt. % colloidal silicon dioxide; 1 wt. % magnesium stearate; 5.4 wt. % cellulose acetate; and 3.6 wt. % polyethylene glycol, based upon the total weight of the tablet formulation.

In some embodiments, the tablet formulation comprises 8.5 wt. % omecamtiv mecarbil dihydrochloride monohydrate; 37.3 wt. % microcrystalline cellulose; 37.3 wt. % lactose monohydrate; 2.6 wt. % hydroxypropyl cellulose; 0.4 wt. % colloidal silicon dioxide; 0.9 wt. % magnesium stearate; 7.8 wt. % cellulose acetate; and 5.2 wt. % polyethylene glycol, based upon the total weight of the tablet formulation.

In some embodiments, the tablet formulation comprises 7.5 wt. % omecamtiv mecarbil dihydrochloride monohydrate; 33 wt. % microcrystalline cellulose; 33 wt. % lactose monohydrate; 2.3 wt. % hydroxypropyl cellulose; 0.4 wt. % colloidal silicon dioxide; 0.8 wt. % magnesium stearate; 13.8 wt. % cellulose acetate; and 9.2 wt. % polyethylene glycol, based upon the total weight of the tablet formulation.

In some embodiments, the core of the tablet formulation comprises 9.8 wt. % omecamtiv mecarbil dihydrochloride monohydrate; 42.8 wt. % microcrystalline cellulose; 42.8 wt. % lactose monohydrate; 3 wt. % hydroxpropyl cellulose; 0.5 wt. % colloidal silicon dioxide; and 1 wt. % magnesium stearate, based upon the total weight of the core; and the film coating results in a 10% coating weight gain for the tablet formulation based upon core total weight, and wherein the film coating comprises 60 wt. % cellulose acetate and 40 wt. % polyethylene glycol, based upon total weight of film coating.

In some embodiments, the core of the tablet formulation comprises 9.8 wt. % omecamtiv mecarbil dihydrochloride monohydrate; 42.8 wt. % microcrystalline cellulose; 42.8 wt. % lactose monohydrate; 3 wt. % hydroxpropyl cellulose; 0.5 wt. % colloidal silicon dioxide; and 1 wt. % magnesium stearate, based upon the total weight of the core; and the film coating results in a 15% coating weight gain for the tablet formulation based upon core total weight, and wherein the film coating comprises 60 wt. % cellulose acetate and 40 wt. % polyethylene glycol, based upon total weight of film coating.

In some embodiments, the core of the tablet formulation comprises 9.8 wt. % omecamtiv mecarbil dihydrochloride monohydrate; 42.8 wt. % microcrystalline cellulose; 42.8 wt. % lactose monohydrate; 3 wt. % hydroxpropyl cellulose; 0.5 wt. % colloidal silicon dioxide; and 1 wt. % magnesium stearate, based upon the total weight of the core; and the film coating results in a 30% coating weight gain for the tablet formulation based upon core total weight, and wherein the film coating comprises 60 wt. % cellulose acetate and 40 wt. % polyethylene glycol, based upon total weight of film coating.

In various embodiments, the omecamtiv mecarbil release profile of the tablet formulation is independent of pH.

### Methods of Making Tablet Formulations

Also provided herein are processes for making a disclosed tablet formulation. As described herein, the process for making the tablet core comprises preparing a granulation comprising one or more the components, after which the granulate is then formed into the tablet core. Any suitable granulation method can be used. The method comprises dry granulation, wet granulation, or a combination thereof.

In some embodiments, the process for making the tablet formulation comprises direct compression of the core components.

In some embodiments, the process for making the tablet formulation comprises admixing the omecamtiv mecarbil, a pharmaceutically acceptable salt thereof, or a pharmaceutically acceptable hydrate of a pharmaceutically acceptable salt thereof, the filler, and the binder and granulating to form a granulated mixture; admixing the granulated mixture and a granulating solvent, and granulating to form a wet granulate; drying the wet granulate to form a dried granulate; milling the dried granulate to form a milled granulate; admixing the milled granulate, the glidant, and the lubricant and compressing the admixture to form the core; admixing the core with a film coating pre-mixture to provide the film coating over the core, and drying the coated core to form the tablet formulation, wherein the film coating pre-mixture comprises the modified-release polymer, the plasticizer, and a film coating solvent.

In some embodiments, the admixing of the omecamtiv mecarbil, filler, and binder is performed with a high shear granulator.

Any suitable granulating solvent(s) can be used in the process to prepare the tablet formulation. In various cases, the granulating solvent is inert and is capable of forming a suitable granulated mixture of components. In an embodiment, the granulating solvent comprises water.

In embodiments of the process comprising milling of a dried granulate, the milling of the dried granulate can be performed using an impact mill.

In some embodiments of the process, the step of admixing of the milled granulate, glidant, and lubricant is performed stepwise such that the milled granulate and glidant are admixed, then the lubricant is admixed with the resulting mixture.

In some embodiments, the process comprises admixing of the core and the film coating pre-mixture in a fluid bed coater.

In some embodiments, the process of making the tablet comprises admixing the omecamtiv mecarbil, a pharmaceutically acceptable salt thereof, or a pharmaceutically acceptable hydrate of a pharmaceutically acceptable salt thereof, the filler, and the binder and granulating to form a granulated mixture; milling the granulated mixture to form a milled granulate; admixing the milled granulate, the glidant, and the lubricant and compressing the admixture to form the core; admixing the core with a film coating pre-mixture to provide the film coating over the core, and drying the coated core to form the tablet formulation, wherein the film coating pre-mixture comprises the modified-release polymer, the pore former, and a film coating solvent.

In some embodiments, the process of making the tablet formulation comprises admixing the omecamtiv mecarbil, or salt or hydrate of a salt thereof, the filler, the binder, the glidant, and the lubricant and compressing the admixture to form the core; admixing the core with a film coating pre-mixture to provide the film coating over the core, and drying the coated core to form the tablet formulation, wherein the film coating pre-mixture comprises the modified-release polymer, the pore former, and a film coating solvent.

Tablet cores as disclosed herein can be prepared by methods including direct compression of core powder blend, wet granulation, or dry granulation of core powder blend followed by rotary compression of granulated blend into cores using multi-tip punches and dies.

### Methods of Use

Also provided herein are methods of use of the disclosed tablet formulations for the treatment of a cardiovascular condition, such as heart failure, including but not limited to acute (or decompensated) congestive heart failure, chronic congestive heart failure, and heart failure with reduced ejection fraction; or a cardiovascular condition associated with systolic heart dysfunction. In certain embodiments, the disclosed tablet formulations can be used in the treatment of heart failure with reduced ejection fraction (HFrEF). In certain embodiments, the disclosed tablet formulations can be used to improve cardiac contractility in a patient suffering from a cardiovascular condition, or to increase ejection fraction in a patient suffering a cardiovascular condition, such as HFrEF.

In further embodiments, the disclosed tablet formulations can be used to (1) improve exercise capacity as determined by cardiopulmonary exercise testing (CPET), (2) improve ventilatory efficiency, as measured by the change in ventilation (VE)/carbon dioxide output (VCO₂) slope during cardiopulmonary exercise testing (CPET), or (3) to improve average daily activity units in a patient suffering from a cardiovascular condition, such as chronic heart failure and heart failure with reduced ejection fraction.

The disclosed methods comprise administering a tablet formulation to a patient in need thereof in a therapeutically effective amount. In some embodiments, the patient is a pediatric patient. In some embodiments, the patient is an adult patient with difficulty swallowing.

In some embodiments, the patient is administered the tablet formulation in an amount to provide 3-50 mg twice daily omecamtiv mecarbil, based upon omecamtiv mecarbil free base weight. In some embodiments, the patient is administered the tablet formulation in an amount to provide 3-25 mg twice daily omecamtiv mecarbil, based upon omecamtiv mecarbil free base weight.

The patient can be a pediatric patient, for example, a child of 6 to 12 years of age. In some cases, the patient is an adult that has difficulty swallowing.

### EMBODIMENTS

1. A tablet formulation comprising:

a core comprising
omecamtiv mecarbil, a pharmaceutically acceptable salt thereof, or a pharmaceutically acceptable hydrate of a pharmaceutically acceptable salt thereof;
a filler;
a binder;
a glidant; and
a lubricant; and

a film coating on the core, the film coating comprising
a modified-release polymer and a pore former.

2. The tablet formulation of embodiment 1, wherein the omecamtiv mecarbil is present as omecamtiv mecarbil dihydrochloride monohydrate.

3. The tablet formulation of embodiment 1 or 2, wherein the filler comprises microcrystalline cellulose, lactose monohydrate, or a combination thereof.

4. The tablet formulation of embodiment 3, wherein the filler comprises microcrystalline cellulose and lactose monohydrate.

5. The tablet formulation of any one of embodiments 1 to 4, wherein the binder comprises hydroxypropyl cellulose.

6. The tablet formulation of any one of embodiments 1 to 5, wherein the glidant comprises silicon dioxide.

7. The tablet formulation of any one of embodiments 1 to 6, wherein the lubricant comprises magnesium stearate.

8. The tablet formulation of any one of embodiments 1 to 7, wherein the core comprises

8-11 wt. % omecamtiv mecarbil, a pharmaceutically acceptable salt thereof, or a pharmaceutically acceptable hydrate of a pharmaceutically acceptable salt thereof;

83-86 wt. % filler;

2-5 wt. % binder;

0.2-0.8 wt. % glidant; and

0.8-1.2 wt. % lubricant.

9. The tablet formulation of any one of embodiments 1 to 8, wherein the film coating further comprises a plasticizer.

10. The tablet formulation of any one of embodiments 1 to 9, wherein the pore former is also a plasticizer.

11. The tablet formulation of any one of embodiments 1 to 10, wherein the modified-release polymer of the film coating comprises ethylcellulose, poly(ethyl acrylate-co-methylmethacrylate), poly(ethyl acrylate-co-methylmethacrylate-co-trimethylammonioethylmethacrylate chloride), cellulose acetate, polyvinyl acetate, or a combination thereof.

12. The tablet formulation of embodiment 11, wherein the modified-release polymer comprises cellulose acetate.

13. The tablet formulation of any one of embodiments 1 to 12, wherein the pore former of the film coating comprises hypromellose, polyvinylpyrrolidone, sorbitol, triethyl citrate, polyethylene glycol, or a combination thereof.

14. The tablet formulation of embodiment 13, wherein the pore former comprises polyethylene glycol.

15. The tablet formulation of embodiment 14, wherein the polyethylene glycol is polyethylene glycol 3350.

16. The tablet formulation of any one of embodiments 9 to 15, wherein the plasticizer comprises polyethylene glycol, diethyl phthalate, triethyl citrate, dibutyl sebacate, triacetin, or a combination thereof.

17. The tablet formulation of any one of embodiments 11 to 16, wherein the film coating comprises

50-90 wt. % modified-release polymer; and

10-50 wt. % pore former and plasticizer (when present).

18. The tablet formulation of embodiment 17, wherein the film coating comprises 60 wt. % modified-release polymer and 40 wt. % pore former and plasticizer (when present).

19. The tablet formulation of any one of embodiments 1 to 18, wherein the film coating comprises 9 wt. % of the total weight of the tablet formulation.

20. The tablet formulation of any one of embodiments 1 to 18, wherein the film coating comprises 13 wt. % of the total weight of the tablet formulation.

21. The tablet formulation of any one of embodiments 1 to 18, wherein the film coating comprises 23 wt. % of the total weight of the tablet formulation.

22. The tablet formulation of embodiment 1, comprising:

5-40 wt. % omecamtiv mecarbil dihydrochloride monohydrate;

10-45 wt. % microcrystalline cellulose;

10-45 wt. % lactose monohydrate;

1-8 wt. % hydroxypropyl cellulose;

0.1-2 wt. % colloidal silicon dioxide;

0.25-3 wt. % magnesium stearate;

3-20 wt. % cellulose acetate; and

2-15 wt. % polyethylene glycol.

23. The tablet formulation of embodiment 22, comprising:

5-10 wt. % omecamtiv mecarbil dihydrochloride monohydrate;

30-45 wt. % microcrystalline cellulose;

30-45 wt. % lactose monohydrate;

1-5 wt. % hydroxypropyl cellulose;

0.1-2 wt. % colloidal silicon dioxide;

0.5-3 wt. % magnesium stearate;

3-20 wt. % cellulose acetate; and

2-15 wt. % polyethylene glycol.

24. The tablet formulation of embodiment 23, comprising:

9 wt. % omecamtiv mecarbil dihydrochloride monohydrate;

38.9 wt. % microcrystalline cellulose;

38.9 wt. % lactose monohydrate;

2.7 wt. % hydroxypropyl cellulose;

0.5 wt. % colloidal silicon dioxide;

1 wt. % magnesium stearate;

5.4 wt. % cellulose acetate; and

3.6 wt. % polyethylene glycol.

25. The tablet formulation of embodiment 23, comprising:

8.5 wt. % omecamtiv mecarbil dihydrochloride monohydrate;

37.3 wt. % microcrystalline cellulose;

37.3 wt. % lactose monohydrate;

2.6 wt. % hydroxypropyl cellulose;

0.4 wt. % colloidal silicon dioxide;

0.9 wt. % magnesium stearate;

7.8 wt. % cellulose acetate; and

5.2 wt. % polyethylene glycol.

26. The tablet formulation of embodiment 23, comprising:

7.5 wt. % omecamtiv mecarbil dihydrochloride monohydrate;

33 wt. % microcrystalline cellulose;

33 wt. % lactose monohydrate;

2.3 wt. % hydroxypropyl cellulose;

0.4 wt. % colloidal silicon dioxide;

0.8 wt. % magnesium stearate;

13.8 wt. % cellulose acetate; and

9.2 wt. % polyethylene glycol.

27. The tablet formulation of embodiment 1, wherein
the core comprises
   9.8 wt. % omecamtiv mecarbil dihydrochloride monohydrate;
   42.8 wt. % microcrystalline cellulose;
   42.8 wt. % lactose monohydrate;
   3 wt. % hydroxpropyl cellulose;
   0.5 wt. % colloidal silicon dioxide; and
   1 wt. % magnesium stearate; and wherein
the film coating results in a 10% coating weight gain for the tablet formulation based upon core total weight, and wherein the film coating comprises 60 wt. % cellulose acetate and 40 wt. % polyethylene glycol, based upon total weight of the film coating.

28. The tablet formulation of embodiment 1, wherein
the core comprises
   9.8 wt. % omecamtiv mecarbil dihydrochloride monohydrate;
   42.8 wt. % microcrystalline cellulose;
   42.8 wt. % lactose monohydrate;
   3 wt. % hydroxpropyl cellulose;
   0.5 wt. % colloidal silicon dioxide; and
   1 wt. % magnesium stearate; and wherein
the film coating results in a 15% coating weight gain for the tablet formulation based upon core total weight, and wherein the film coating comprises 60 wt. % cellulose acetate and 40 wt. % polyethylene glycol, based upon total weight of the film coating.

29. The tablet formulation of embodiment 1, wherein
the core comprises
   9.8 wt. % omecamtiv mecarbil dihydrochloride monohydrate;
   42.8 wt. % microcrystalline cellulose;
   42.8 wt. % lactose monohydrate;
   3 wt. % hydroxpropyl cellulose;
   0.5 wt. % colloidal silicon dioxide; and
   1 wt. % magnesium stearate; and wherein
the film coating results in a 30% coating weight gain for the tablet formulation based upon core total weight, and wherein the film coating comprises 60 wt. % cellulose acetate and 40 wt. % polyethylene glycol, based upon total weight of the film coating.

30. The tablet formulation of any one of embodiments 1 to 29, comprising 1-3 mg omecamtiv mecarbil.

31. The tablet formulation of embodiment 30, comprising 1 mg omecamtiv mecarbil.

32. The tablet formulation of any one of embodiments 1 to 24, 27, 30, and 31, having an omecamtiv mecarbil release profile of:
less than or equal to 50% omecamtiv mecarbil released at 1 hour;
60-70% omecamtiv mecarbil released at 2 hours;
85-90% omecamtiv mecarbil released at 8 hours; and
greater than or equal to 90% omecamtiv mecarbil released at 16 hours.

33. The tablet formulation of any one of embodiments 1 to 23, 25, 28, 30, and 31, having an omecamtiv mecarbil release profile of:
less than or equal to 25% omecamtiv mecarbil released at 1 hour;
35-45% omecamtiv mecarbil released at 2 hours;
75-80% omecamtiv mecarbil released at 8 hours; and
greater than or equal to 85% omecamtiv mecarbil released at 16 hours.

34. The tablet formulation of any one of embodiments 1 to 23, 26, and 29 to 31, having an omecamtiv mecarbil release profile of:
less than or equal to 10% omecamtiv mecarbil released at 1 hour;
25-35% omecamtiv mecarbil released at 2 hours;
70-75% omecamtiv mecarbil released at 8 hours; and
greater than or equal to 78% omecamtiv mecarbil released at 16 hours.

35. The tablet formulation of any one of embodiments 1-34, wherein upon administration to a patient, the tablet formulation provides a maximum plasma concentration (Cₘₐₓ) of omecamtiv mecarbil in the patient of 100-1000 ng/mL 36. The tablet formulation of any one of embodiments 1 to 35, not comprising (free of) a pH-modifying agent.

37. The tablet formulation of any one of embodiments 1 to 36 having a diameter of up to 3 mm.

38. A method of treating heart failure in a patient suffering therefrom, comprising administering to the patient the tablet formulation of any one of embodiments 1 to 37.

39. The method of embodiment 38, wherein the heart failure is acute or chronic.

40. The method of embodiment 38, wherein the heart failure is heart failure with reduced ejection fraction (HFrEF).

41. The method of any one of embodiments 38 to 40, wherein the patient is a pediatric patient.

42. The method of embodiment 41, wherein the pediatric patient is administered the tablet formulation in an amount to provide 3-25 mg twice daily omecamtiv mecarbil.

43. The method of any one of embodiments 38 to 40, wherein the patient is an adult patient with difficulty swallowing.

44. The method of embodiment 43, wherein the adult patient is administered the tablet formulation in an amount to provide 25 mg or 50 mg twice daily omecamtiv mecarbil.

45. The tablet formulation of any one of embodiments 1 to 37 for use in treating heart failure.

46. The tablet formulation of embodiment 45, wherein the heart failure is acute or chronic.

47. The tablet formulation of embodiment 45, wherein the heart failure is heart failure with reduced ejection fraction (HFrEF).

48. The tablet formulation of any one of embodiments 45 to 47, wherein the tablet formulation is suitable for administration to a pediatric patient.

49. The tablet formulation of embodiment 48, wherein the pediatric patient is administered the tablet formulation in an amount to provide 3-25 mg twice daily omecamtiv mecarbil.

50. The tablet formulation of any one of embodiments 45 to 47, wherein the tablet formulation is suitable for administration to an adult patient with difficulty swallowing.

51. The tablet formulation of embodiment 50, wherein the adult patient is administered the tablet formulation in an amount to provide 25 mg or 50 mg twice daily omecamtiv mecarbil.

52. Use of the tablet formulation of any one of embodiments 1 to 37 in the preparation of a medicament for the treatment of heart failure.

53. The use of embodiment 52, wherein the heart failure is acute or chronic.

54. The use of embodiment 52, wherein the heart failure is heart failure with reduced ejection fraction (HFrEF).

55. The use of any one of embodiments 52 to 54, wherein the tablet formulation is suitable for administration to a pediatric patient.

56. The use of embodiment 55, wherein the pediatric patient is administered the tablet formulation in an amount to provide 3-25 mg twice daily omecamtiv mecarbil.

57. The use of any one of embodiments 52 to 54, wherein the tablet formulation is suitable for administration to an adult patient with difficulty swallowing.

58. The use of embodiment 57, wherein the adult patient is administered the tablet formulation in an amount to provide 25 mg or 50 mg twice daily omecamtiv mecarbil.

59. A process for making the tablet formulation of any one of embodiments 1 to 37, comprising:
admixing the omecamtiv mecarbil, a pharmaceutically acceptable salt thereof, or a pharmaceutically acceptable hydrate of a pharmaceutically acceptable salt thereof, the filler, and the binder and granulating to form a granulated mixture;
admixing the granulated mixture and a granulating solvent, and granulating to form a wet granulate;
drying the wet granulate to form a dried granulate;
milling the dried granulate to form a milled granulate;
admixing the milled granulate, the glidant, and the lubricant and compressing the admixture to form the core;
admixing the core with a film coating pre-mixture to provide the film coating over the core, and
drying the coated core to form the tablet formulation,
   wherein the film coating pre-mixture comprises the modified-release polymer, the plasticizer, and a film coating solvent.

60. The process of embodiment 59, wherein the admixing of the omecamtiv mecarbil, a pharmaceutically acceptable salt thereof, or a pharmaceutically acceptable hydrate of a pharmaceutically acceptable salt thereof, the filler, and binder is performed with a high shear granulator.

61. The process of embodiment 59 or 60, wherein the granulating solvent comprises water.

62. The process of any one of embodiments 59 to 61, wherein the milling of the dried granulate is performed using an impact mill.

63. The process of any one of embodiments 59 to 62, wherein the admixing of the milled granulate, the glidant, and the lubricant is performed stepwise such that the milled granulate and the glidant are admixed, then the lubricant is admixed with the resulting mixture.

64. The process of any one of embodiments 59 to 63, wherein the film coating solvent comprises acetone, water, or a mixture thereof.

65. The process of embodiment 64, wherein the film coating solvent comprises acetone and water.

66. The process of embodiment 65, wherein the film coating solvent comprises 9:1 acetone:water.

67. The process of any one of embodiments 59 to 66, wherein the admixing of the core and the film coating pre-mixture is performed in a fluid bed coater.

68. A process of making the tablet formulation of any one of embodiments 1 to 37, comprising
admixing the omecamtiv mecarbil, a pharmaceutically acceptable salt thereof, or a pharmaceutically acceptable hydrate of a pharmaceutically acceptable salt thereof, the filler, and the binder and granulating to form a granulated mixture;
milling the granulated mixture to form a milled granulate;
admixing the milled granulate, the glidant, and the lubricant and compressing the admixture to form the core;
admixing the core with a film coating pre-mixture to provide the film coating over the core, and
drying the coated core to form the tablet formulation,
   wherein the film coating pre-mixture comprises the modified-release polymer, the plasticizer (when present), and a film coating solvent.

69. A process of making the tablet formulation of any one of embodiments 1 to 37, comprising
admixing the omecamtiv mecarbil, a pharmaceutically acceptable salt thereof, or a pharmaceutically acceptable hydrate of a pharmaceutically acceptable salt thereof, the filler, the binder, the glidant, and the lubricant and compressing the admixture to form the core;
admixing the core with a film coating pre-mixture to provide the film coating over the core, and
drying the coated core to form the tablet formulation,
   wherein the film coating pre-mixture comprises the modified-release polymer, the plasticizer (when present), and a film coating solvent.

### EXAMPLES

The following examples further illustrate the disclosed tablet formulation and process, but of course, should not be construed as in any way limiting its scope.

The following abbreviations are used in the Examples: IR refers to immediate-release; OM refers to omecamtiv mecarbil; MCC refers to microcrystalline cellulose; HPC refers to hydroxypropyl cellulose; CA refers to cellulose acetate; PEG refers to polyethylene glycol; SSNMR refers to solid state nuclear magnetic resonance; AV refers to acceptance value; PK refers to pharmacokinetics; GLSM refers to geometric least squares mean; CI refers to confidence interval; and CV refers to coefficient of variation.

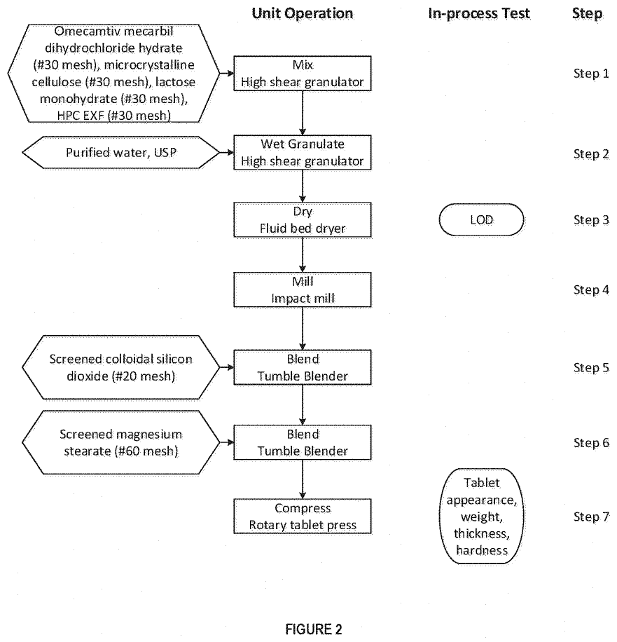

An exemplary process for manufacturing immediate-release mini-tablet cores is depicted in FIG. 2. The illustrative process comprises the following steps: 1) mixing the screened intra-granular components in a high shear wet granulator; 2) granulating the intra-granular components while delivering purified water; 3) drying the wet material to a pre-defined Loss-on-Drying value (LOD); 4) milling the dried granules using an impact mill; 5) blending the milled granules with pre-screened colloidal silicon dioxide in a tumble blender; 6) blending the product of step 5) with pre-screened magnesium stearate in a tumble blender; and 7) compressing the final blend using a rotary tablet press, wherein the tablet appearance, weight, thickness, and hardness are monitored throughout the compression process.

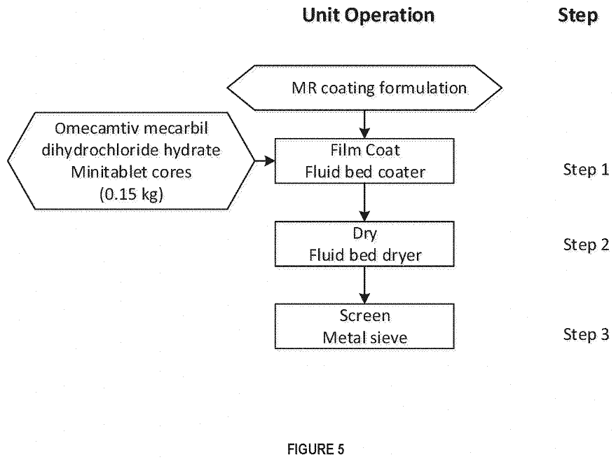

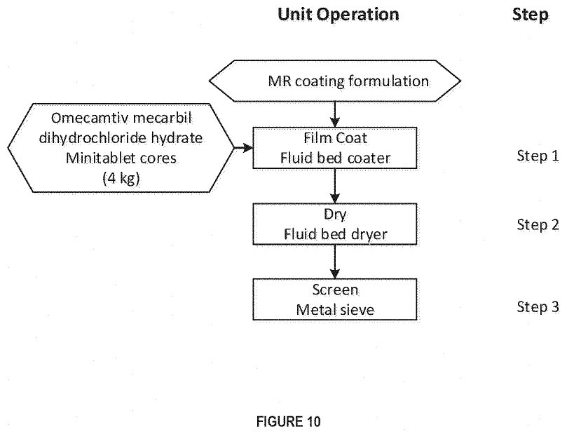

An exemplary process for coating the immediate-release mini-tablet cores is depicted in FIGS. 5 and 10. The illustrative process comprises the following steps: 1) film coating the mini-tablet cores with the MR coating in a fluid bed coater; 2) drying the coated mini-tablets in a fluid bed dryer; and 3) screening the dried mini-tablets through a sieve. The illustrative coating process was conducted on either a 0.15 kg scale (FIG. 5), or on a 4 kg scale (FIG. 10).

### Example 1

Mini-tablet cores. This example demonstrates omecamtiv mecarbil dihydrochloride monohydrate containing cores in accordance with an aspect of the disclosed tablet formulations. In particular, this example illustrates mini-tablet cores not comprising (free of) a pH modifier and cores comprising a pH modifier.

Three tablet cores (Cores 1A-1C) were prepared comprising the components listed in Table 1 using the procedure described in the general Example section above. Intra-granular components consisted of omecamtiv mecarbil dihydrochloride monohydrate, microcrystalline cellulose (MCC, Avicel PH 101) and lactose monohydrate (impalpable 313), hydroxypropyl cellulose (HPC, Klucel EXF), and optionally fumaric acid (Cores 1B and 1C). Extra-granular components consisted of colloidal silicon dioxide and magnesium stearate.

The mini-tablet cores were prepared with varying amounts of fumaric acid as a pH modifier. While Core 1A was substantially free of fumaric acid, Core 1B had a 1:1 ratio by weight of fumaric acid to omecamtiv mecarbil and Core 1C had a 2:1 ratio by weight of fumaric acid to omecamtiv mecarbil.

TABLE 1
Omecamtiv mecarbil dihydrocloride
monohydrate 1 mg IR mini-tablet cores

| | | Core 1A | Core 1B | Core 1C |
|---|---|---|---|---|
| Material | | Theo. w/w %, 1 mg | | |
| OM dihydrochloride | | 9.812 | 9.812 | 9.812 |
| monohydrate^{a} | | | | |
| Fumaric acid | | 0.000 | 9.812 | 19.625 |
| MCC, Avicel PH 101 | | 42.844 | 37.938 | 33.0315 |
| Lactose monohydrate, | | 42.844 | 37.938 | 33.0315 |
| impalpable 313 | | | | |
| HPC, Klucel EXF | | 3.000 | 3.000 | 3.000 |
| Colloidal silicon dioxide | | 0.500 | 0.500 | 0.500 |
| Magnesium stearate | | 1.000 | 1.000 | 1.000 |
| Core Tablet Total | | 100.0 | 100.0 | 100.0 |
| Core Target | | 12.5 | | |
| Weight (mg) | | | | |
| Core Target | | 2.0 | | |
| Hardness (kp) | | | | |
| Observed weight | | 12.6 ± 0.18 | 12.3 ± 0.19 | 12.3 ± 0.22 |
| (mg), n ≥ 10 | | | | |
| Observed hardness | | 1.8 ± 0.25 | 1.9 ± 0.32 | 1.9 ± 0.25 |
| (kp), n ≥ 10 | | | | |
| Content | Mean | 99.7 | 99.6 | 100.8 |
| Uniformity | (Min-Max) | (92.2-105) | (96.7-103.2) | (98-104.3) |
| | AV ≤ 15 | 8.4 | 4.7 | 4.1 |

^{a}Drug substance in the table formulation was adjusted according to the “Theoretical free base equivalent”: 81.53%.

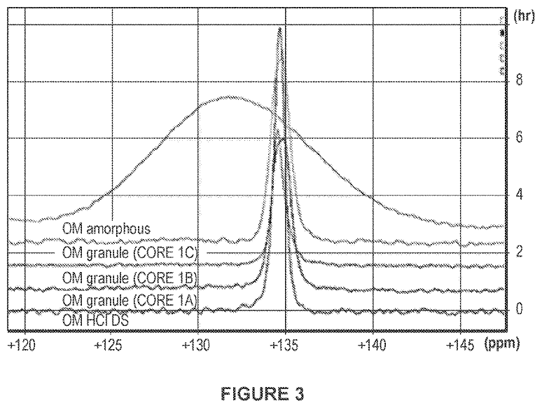

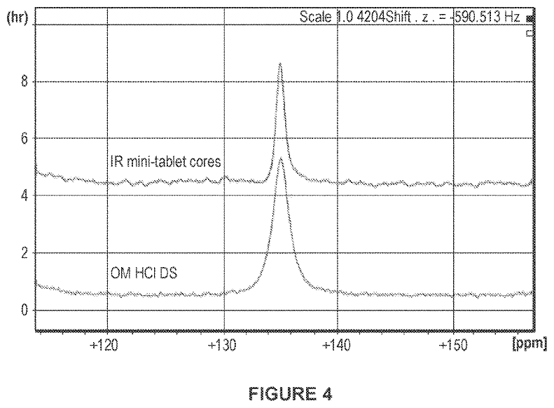

Cores 1A-1C, as well as the corresponding granulates, were subjected to analytical characterizations. The results are depicted in FIGS. 3 and 4.

FIGS. 3 and 4 depict the ¹⁹F solid state NMR (SSNMR) spectra of the immediate-release granulation and immediate-release mini-tablet cores. The results demonstrate that omecamtiv mecarbil dihydrochloride monohydrate is stable during manufacturing and exhibits no change in physical form during the manufacturing process.

### Example 2

Modified-release coating. This example demonstrates a modified-release coating in accordance with an aspect of the disclosed tablet formulations.

The tablet cores from Example 1 (Core 1A-1C) were coated with a modified-release (MR) coating comprising 70:30 CA:PEG to various target coating weight gains (i.e., 10%, 15%, or 20% weight gain). The MR coatings containing cellulose acetate and polyethylene glycol were prepared as a 5 wt. % solution in acetone:water (9:1). The cores were coated using a fluid bed coater.

The dissolution profiles of the MR-coated mini-tablets were determined by the U.S. Pharmacopeia (USP) II method using the following parameters: apparatus is USP <711> Apparatus II (paddle); vessel size/type is a 1000 mL clear glass, round bottom; rotation speed is 75 rpm; media volume is 500 mL; test temperature is 37.0±0.5° C.; dissolution media is phosphate buffer (pH 6.8); and sampling time points are 1, 2, 3, 4, 6, 8, 12, 16, and 24 hours. The test solutions are assayed using high performance liquid chromatography (HPLC) using the following conditions: pump is isocratic; reverse-phase column (for example, X-Bridge, 150×3 mm (id), C18, 3.5 μm particle size, commercially available from Waters); UV detection (235 nm); injection volume of 75 μL; flow rate of 0.5 mL/min; column temperature is 30° C.; autosampler temperature is ambient temperature; and a run time of 6 minutes.

The results are shown in FIGS. 6-8.

FIG. 6 shows the dissolution profiles of immediate-release mini-tablet cores containing 1 mg omecamtiv mecarbil monohydrate coated with 70:30 CA:PEG to 10% coating weight gain in pH 6.8 buffer, respectively. The dissolution profiles show that fumaric acid in the immediate-release mini-tablet core did not enhance omecamtiv mecarbil release in pH 6.8 buffer.

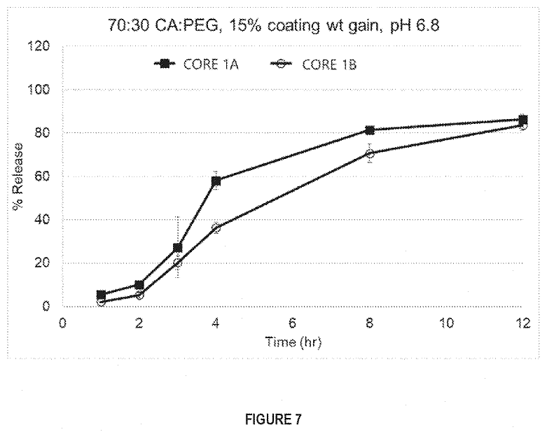

FIG. 7 shows the dissolution profiles of immediate-release mini-tablet cores containing 1 mg omecamtiv mecarbil monohydrate coated with 70:30 CA:PEG to 15% coating weight gain in pH 6.8 buffer, respectively. The dissolution profiles show that fumaric acid in mini-tablet core did not enhance omecamtiv mecarbil release in pH 6.8 buffer.

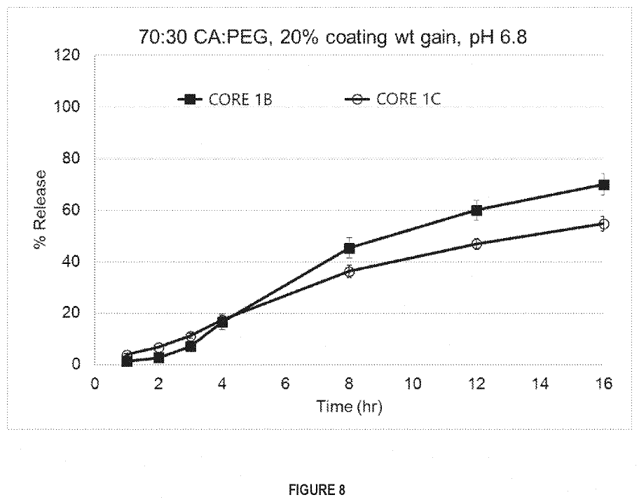

FIG. 8 shows the dissolution profiles of immediate-release mini-tablet cores containing 1 mg omecamtiv mecarbil monohydrate coated with 70:30 CA:PEG to 20% coating weight gain in pH 6.8 buffer, respectively. The dissolution profiles show that fumaric acid did not enhance omecamtiv mecarbil release in pH 6.8 buffer.

The dissolution studies results suggest that fumaric acid is not required in the immediate-release mini-tablet core.

### Example 3

Modified-release coating. This example demonstrates a modified-release coating in accordance with an aspect of the disclosed tablet formulations.

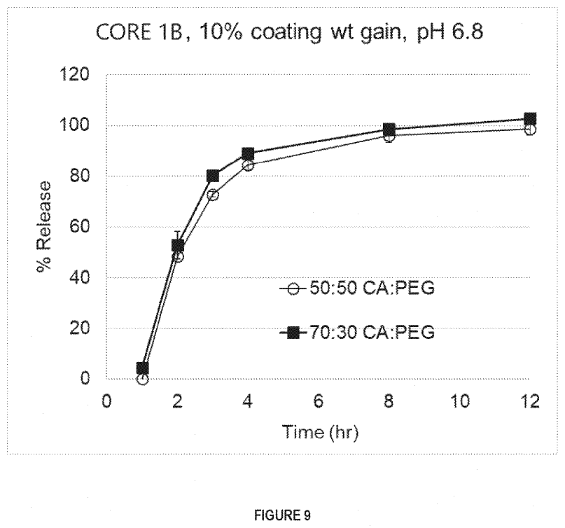

Tablet Core 1B from Example 1 was coated with two different MR coatings comprising cellulose acetate and PEG to a 10% weight gain. The coating compositions had a CA:PEG ratio of either 70:30 or 50:50. The dissolution profiles of the MR-coated mini-tablets were determined using the method described in Example 2. The results of the dissolutions studies are depicted in FIG. 9.

As shown in FIG. 9, the dissolution profiles show that the coating comprising 50:50 CA:PEG to a 10% weight gain did not increase omecamtiv mecarbil release significantly compared with the coating comprising 70:30 CA:PEG. Without wishing to be bound to any particular theory, it is believed that this result would not be observed at higher coating weight gains. At the same and higher coating weight gain, it is expected that omecamtiv mecarbil will release faster with the 50:50 CA:PEG than 70:30 CA:PEG coating, therefore omecamtiv mecarbil release rate will be less sensitive to coating weight gain with the coating comprising 50:50 CA:PEG. Being less sensitive to the coating weight gain with the coating comprising 50:50 CA:PEG allows for release rate robustness. However, more coating weight gain is required to achieve a slower release rate using the 50:50 CA:PEG coating. More coating weight gain leads to low manufacturing efficiency because more coating weight gain will require longer coating time.

To balance the coating process efficiency and release rate robustness and also in reference to the dissolution profiles presented in FIGS. 6-9, a coating comprising 60:40 CA:PEG was selected to coat omecamtiv mecarbil immediate-release mini-tablets at different coating weight gains to achieve the target omecamtiv mecarbil release profiles.

### Example 4

Tablet formulation. This example demonstrates an embodiment of the disclosed tablet formulations.

Tables 2 and 3 show a tablet formulation comprising an immediate-release mini-tablet core and MR coating. The tablet core was prepared according to the process shown in FIG. 2, and the cores were coated using the process shown in FIG. 10.

TABLE 2
Omecamtiv mecarbil immediate-release mini-tablet cores

| | IR Mini-Tablet (1 mg) | |
|---|---|---|
| Material | Theo, w/w % | Theo, mg/unit |
| OM dihydrochloride monohydrate ^{a} | 9.812 | 1.227 |
| MCC, Avicel PH 101 | 42.848 | 5.356 |
| Lactose monohydrate impalpable 313 | 42.840 | 5.355 |
| Hydroxypropyl cellulose, Klucel EXF | 3.000 | 0.375 |
| Colloidal silicon dioxide | 0.500 | 0.0625 |
| Magnesium stearate | 1.000 | 0.125 |
| Core Tablet Total | 100.0 | 12.5 |

^{a} Drug substance in the core was adjusted according to the “Theoretical free base equivalent”: 81.53%.

TABLE 3
MR film-coating composition for
omecamtiv mecarbil IR mini-tablet cores

| Component | Percent (% w/w) |
|---|---|
| Cellulose Acetate (CA) | 60:40 |
| Polyethylene Glycol (PEG) 3350 | |

Note:
acetone and water are used as coating solvent at 90:10 weight ratio. MR coating pre-mixture contained 5-10% solids and 90-95% solvent by weight.

### Example 5

In-vitro release rates. Tablet formulations with three in vitro release rates were developed by coating immediate-release mini-tablet cores with MR coating to three different weight gains. Two batches (10 kg and 20 kg) of immediate-release mini-tablet cores were prepared using the process depicted in FIG. 2. As shown in Tables 4A and 4B, the immediate-release mini-tablet cores of Example 4 were coated using the process depicted in FIG. 10 with the MR coating to a target weight gain of 10%, 15%, or 30% to achieve fast, medium and slow release of omecamtiv mecarbil, respectively. Table 4a lists the weight percentages relative to the weight of the core. Table 4b lists the weight percentages relative to the total weight of the tablet formulation.

TABLE 4A
MR tablet formulations comprising omecamtiv mecarbil (wt. % relative to core)

| | 1 mg | | 1 mg | | 1 mg | |
|---|---|---|---|---|---|---|
| | (Fast release) | | (Medium release) | | (Slow release) | |
| Component | (% w/w) | (mg/tab) | (% w/w) | (mg/tab) | (% w/w) | (mg/tab) |
| | Intra-granular | | | | | |
| CM dihydrochloride | 9.8 | 1.2 | 9.8 | 1.2 | 9.8 | 1.2 |
| monohydrate^{a} | (8.0) | (1.0) | (8.0) | (1.0) | (8.0) | (1.0) |
| (Free base) | | | | | | |
| MCC, Avicel PH101 | 42.8 | 5.4 | 42.8 | 5.4 | 42.8 | 5.4 |
| Lactose monohydrate, | 42.8 | 5.4 | 42.8 | 5.4 | 42.8 | 5.4 |
| Impalpable 313 | | | | | | |
| HPC, Klucel EXF | 3.0 | 0.4 | 3.0 | 0.4 | 3.0 | 0.4 |
| Purified water^{b} | — | — | — | — | — | — |
| | Extra-granular | | | | | |
| Colloidal silicon dioxide | 0.5 | 0.06 | 0.5 | 0.06 | 0.5 | 0.06 |
| Magnesium stearate | 1.0 | 0.1 | 1.0 | 0.1 | 1.0 | 0.1 |
| Core Mini-Tablet Total | 99.9 | 12.6 | 99.9 | 12.6 | 99.9 | 12.6 |
| | MR film coating | | | | | |
| Cellulose acetate (CA) 398-10 | 6.0 | 0.8 | 9.0 | 1.1 | 18.0 | 2.3 |
| Polyethylene glycol (PEG)3350 | 4.0 | 0.5 | 6.0 | 0.8 | 12.0 | 1.5 |
| Acetone^{b} | — | — | — | — | — | — |
| Purified water^{b} | — | — | — | — | — | — |

^{a}The molecular weight of dihydrochloride monohydrate salt and free base are 492.37 and 401.43 g/mol, respectively, the guantity used may be adjusted by the assay of the drug substance batch.
^{b}Evaporated during the manufacturing process.

TABLE 4B
MR tablet formulations comprising omecamtiv mecarbil (wt. % relative to total tablet formulation)

| | 1 mg | | 1 mg | | 1 mg | |
|---|---|---|---|---|---|---|
| | (Fast release) | | (Medium Release) | | (Slow release) | |
| Component | (% w/w) | (mg/tab) | (% w/w) | (mg/tab) | (% w/w) | (mg/tab) |
| | Intra-granular | | | | | |
| CM dihydrochloride | 8.9 | 1.2 | 8.5 | 1.2 | 7.6 | 1.2 |
| monohydrate^{a} | (7.2) | (1.0) | (6.9) | (1.0) | (6.1) | (1.0) |
| (Free base) | | | | | | |
| MCC, Avicel PH101 | 38.9 | 5.4 | 37.3 | 5.4 | 33.0 | 5.4 |
| Lactose monohydrate, | 38.9 | 5.4 | 37.3 | 5.4 | 33.0 | 5.4 |
| Impalpable 313 | | | | | | |
| HPC, Klucel EXF | 2.7 | 0.4 | 2.6 | 0.4 | 2.3 | 0.4 |
| Purified water^{b} | — | — | — | — | — | — |
| | Extra-granular | | | | | |
| Colloidal silicon dioxide | 0.5 | 0.06 | 0.4 | 0.06 | 0.4 | 0.06 |
| Magnesium stearate | 0.9 | 0.1 | 0.9 | 0.1 | 0.8 | 0.1 |
| | MR film coating | | | | | |
| Cellulose acetate (CA) 398-10 | 5.5 | 0.8 | 7.8 | 1.1 | 13.8 | 2.3 |
| Polyethylene glycol (PEG)3350 | 3.6 | 0.5 | 5.2 | 0.8 | 9.2 | 1.5 |
| Acetone^{b} | — | — | — | — | — | — |
| Purified water^{b} | — | — | — | — | — | — |

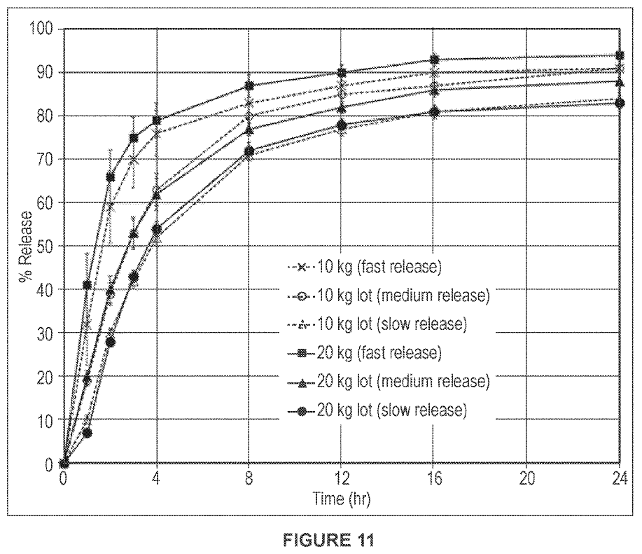

Tables 5 and 6 show the characterization data of immediate-release mini-tablet cores and MR-coated mini-tablets for both batches. The data shows that both immediate-release mini-tablet cores and MR-coated mini-tablet meet the target tablet weight, tablet hardness, assay, and uniformity values. FIG. 11 shows a comparison of the three release rates (fast, medium, and slow) from the two batches. These results demonstrated consistent release rates from both batches.

TABLE 5
OM dihydrochloride monohydrate 1 mg IR mini-tablet cores

| Property | (10 kg) | (20 kg) |
|---|---|---|
| Target Weight (mg) | 12.5 | |
| Avg. Tablet Weight (mg) | 12.5 ± 0.12 | 12.6 ± 0.20 |
| | (12.3-12.8, n = 75) | (12.1-13.2, n = 305) |
| Avg. Tablet Thickness (mm) | 2.4 ± 0.02 | 2.4 ± 0.02 |
| | (2.3-2.4, n = 75)) | (2.3-2.4, n = 305) |
| Target Hardness (kp) | 3.2 | |
| Avg. tablet Hardness (kP) | 3.2 ± 0.33 | 3.1 ± 0.26 |
| | (2.5-3.9, n = 75) | (2.6-4.1, n = 305) |

TABLE 6
Characterization of OM dihydrochloride
monohydrate 1 mg MR mini-tablets

| | | Assay, % label | |
|---|---|---|---|
| Batch | Lot | claim (Target: | Content uniformity |
| name | number | 90% to 110%) | (Target: AV ≤ 15.0) |
| 10 kg | Fast | 97.6 | Mean: 97.5%, AV: 5.5 |
| | Medium | | Mean: 97.8%, AV: 2.1 |
| | Slow | | Mean: 98.0%, AV: 4.7 |
| 20 kg | Fast | 99.2 | Mean: 98.5%, AV: 3.0 |
| | Medium | 100.2 | Mean: 99.8%, AV: 3.8 |
| | Slow | 100.3 | Mean: 98.6%, AV: 5.2 |

### Example 6

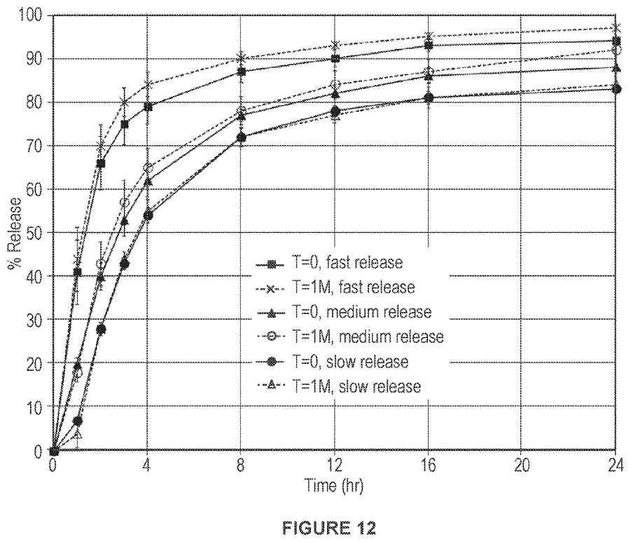

Stability Study. The MR mini-tablets prepared in Example 5 were packaged in 45 cc HDPE bottles (150 counts) with heat induction seal and polypropylene CRCs. Drug product stability was monitored under long term (5° C.) and accelerated (25° C./60% RH) for 24 months. The stability results up to one month are summarized in Table 7 and FIG. 12.

The assay for impurities was done using HPLC under the following conditions: reverse-phase column (for example, X-Bridge, 150×3 mm (id), C18, 3.5 μm particle size, commercially available from Waters); UV detection (235 nm); injection volume of 20 μL; flow rate of 0.45 mL/min; column temperature is 30° C.; autosampler temperature is ambient temperature; and a run time of 26 minutes using the following gradient conditions:

| | Mobile | Mobile |
|---|---|---|
| | Phase | Phase |
| Time (min) | A (%) | B (%) |
| 0 | 92 | 8 |
| 3.0 | 92 | 8 |
| 15.5 | 50 | 50 |
| 18.5 | 20 | 80 |
| 20.5 | 20 | 80 |
| 20.6 | 92 | 8 |
| 26.0 | 92 | 8 |

wherein mobile phase A is 0.2% ammonium hydroxide in water (for example, 2 mL of ammonium hydroxide and diluted to 1000 mL with purified, filtered water, Milli-Q). Mobile phase B is 0.2% ammonium hydroxide in acetonitrile (for example, 2 ml of ammonium hydroxide and diluted to 1000 mL with acetonitrile).

The results show no significant changes in appearance, assay, water content, and dissolution after 1 month under the storage conditions.

TABLE 7

| | | 25° C./60% RH | | | | | | |
|---|---|---|---|---|---|---|---|---|
| | | Fast release | | | Medium release | | Slow release | |
| Test | Specification | T = 0 | | T = 1M | T = 0 | T = 1M | T = 0 | T = 1M |
| Appearance | white to off-white round convex mini-tablet | Conform | | Conform | Conform | Conform | Conform | Conform |
| | with no obvious physical defects | | | | | | | |
| Assay by HPLC | 90.0-110.0 (% Label Claim) | 99.2 | | 99.2 | 100.2 | 99.0 | 100.3 | 99.2 |
| Impurities by HPLC | | % (RRT) | | | | | | |
| • Specified | 5-amino-2-methylpyridine | ≤0.5% | ND | <0.05 | ND | <0.05 | ND | <0.05 |
| Impurities | | | (0.29) | | (0.29) | | (0.29) | |
| | methyl 4-(3-amino-2-fluorobenzyl) | ≤0.5% | <0.05 | <0.05 | <0.05 | <0.05 | <0.05 | <0.05 |
| | piperazine-1-carboxylate | | (0.90) | | (0.90) | | (0.90) | |
| | 1-(2-fluoro-3-(3-(6-methylpyridin-3-yl)ureido) | ≤3.0% | ND | ND | ND | ND | ND | ND |
| | benzyl-4-(methoxycarbonyl)piperazine 1-oxide | | (0.72) | (0.72) | (0.72) | (0.72) | (0.72) | (0.72) |
| | aldehyde (1-(2-fluoro-3-formylphenyl)- | ≤1.0% | ND | ND | ND | ND | ND | ND |
| | 3-(6-methylpyridin-3-yl)urea | | (0.94) | (0.94) | (0.94) | (0.94) | (0.94) | (0.94) |

| | | | | | | | |
|---|---|---|---|---|---|---|---|
| • Unspecified | ≤0.5% | <0.05 | — | <0.05 | — | <0.05 | — |
| Impurities | | (0.69) | | (0.69) | | (0.69) | |
| | | 0.06 | — | 0.06 | — | 0.06 | — |
| | | (0.81) | | (0.81) | | (0.81) | |
| | | <0.05 | — | <0.05 | — | <0.05 | — |
| | | (1.19) | | (1.19) | | (1.19) | |
| | | <0.05 | — | <0.05 | — | <0.05 | — |
| | | (1.21) | | (1.21) | | (1.21) | |
| | | <0.05 | — | <0.05 | — | <0.05 | — |
| | | (1.26) | | (1.26) | | (1.26) | |
| | | ND | 0.18 | ND | 0.20 | ND | 0.18 |
| | | (1.24) | (1.24) | (1.24) | (1.24) | (1.24) | (1.24) |
| Total Impurity | ≤5.0% | 0.06 | 0.18 | 0.06 | 0.20 | 0.06 | 0.18 |
| Water content | Report (%) | 3.14 | 4.10 | 3.16 | 4.00 | 3.26 | 3.59 |
| by Karl Fischer | | | | | | | |

### Example 7

In vivo studies. The primary objective of the study was to evaluate the pharmacokinetics (PK) of OM following single doses of the following in healthy adult subjects: 25 mg (1×25 mg) modified-release (MR) tablet formulation, 25 mg (25×1 mg) slow release mini-tablet formulation, 25 mg (25×1 mg) fast-release mini-tablet formulation, 6 mg (6×1 mg) slow-release mini-tablet formulation, and 6 mg (6×1 mg) fast release mini-tablet formulation.

The secondary objective of the study was to evaluate the safety and tolerability of a single 6 mg dose (administered as mini-tablets) or 25 mg dose (administered as a MR tablet or mini-tablets) of OM administered to healthy adult subjects.

The study was a Phase 1, single-center, open-label, randomized, 5-period, 4-sequence crossover study to investigate the pharmacokinetics, safety, and tolerability of the disclosed tablet formulations and a conventional MR tablet formulation of OM in healthy male and healthy female adult subjects. Subjects were screened to assess eligibility to enter the study within 21 days prior to the first dose administration. Subjects were admitted into the clinical research unit on Day 1 and confined to the clinical research unit for Periods 1 through 5 until discharged at end of study. Following randomization into 1 of 4 treatment sequences, subjects received 1 of the 5 treatments on Day 1 of each treatment period with all subjects receiving all 5 treatments. Blood was collected at predetermined timepoints to characterize plasma concentrations of OM. Safety and tolerability monitoring was performed throughout the study.

### Study Design

Up to twenty subjects (5 per sequence) were enrolled in the study. Data for all 20 subjects entered into the study were included in the PK and safety analyses. Healthy male or female subjects were selected according to inclusion and exclusion criteria. The subjects were assigned to one of the five following Treatment Groups (A-E):
Treatment Group A: 25 mg (1×25 mg) OM MR tablet;
Treatment Group B: 25 mg (25×1 mg) OM slow-release mini-tablets;
Treatment Group C: 25 mg (25×1 mg) OM fast-release mini-tablets;
Treatment Group D: 6 mg (6×1 mg) OM slow-release mini-tablets; and
Treatment Group E: 6 mg (6×1 mg) OM fast-release mini-tablets.

Doses were administered orally after an overnight fast of at least 10 hours with approximately 8 ounces (240 mL) of water. Subjects received instructions that the tablets were not to be broken or chewed and that dosing of all mini-tablets was to be completed in less than 5 minutes. Single oral doses were administered on Day 1 of each treatment period with a minimum washout of 7 days between treatments. All subjects received a single dose of each of the 5 treatments.

### Pharmacokinetics

Blood samples were collected for the analysis of plasma concentrations of OM. The PK parameters determined from the plasma concentrations of OM were as follows: maximum plasma concentration (Cₘₐₓ), area under the plasma concentration-time curve (AUC) from time zero to the time of the last quantifiable concentration (AUCₗₐₛₜ), AUC from time zero to infinity (AUC_{inf}), time of the maximum plasma concentration (tₘₐₓ), and apparent terminal elimination half-life (t_{1/2}), percentage of AUC_{inf} that is due to extrapolation from the time of last measurable concentration to infinity (% AUCₑₓₜᵣₐₚ), elimination rate constant (λ_{z}), correlation coefficient of terminal elimination phase (R²); number of data points included in determination of λ_{z} (number of points), difference between start and end of exponential fit divided by t_{1/2} (λ_{z} Span ratio), lower limit of the terminal phase (start of exponential fit), upper limit of the terminal phase (end of exponential fit).

### Safety

The safety analysis included monitoring adverse events, clinical laboratory evaluations, 12 lead electrocardiograms (ECGs), and vital signs during the study.

Single doses of OM were safe and well tolerated by healthy adult subjects when administered as 25 mg (1×25 mg) MR tablet formulation, 25 mg (25×1 mg) slow-release mini-tablet formulation, 25 mg (25×1 mg) fast-release mini-tablet formulation, 6 mg (6×1 mg) slow-release mini-tablet formulation, and 6 mg (6×1 mg) fast-release mini-tablet formulation were safe and well tolerated when administered to healthy subjects in this study. All adverse events were mild in severity and resolved by the end of the study. There were no serious adverse events and no treatment-emergent adverse events led to premature discontinuation of a subject from the study. Eight treatment-emergent adverse events were reported by 7 subjects that were considered by the investigator to be related to OM including myalgia, dizziness, costochondritis, muscle twitching, and non-cardiac chest pain. There were no clinically significant findings in clinical laboratory evaluations, vital signs, or 12-lead ECGs during the study.

### Statistical Analysis

A statistical analysis was conducted to investigate the bioavailability on the PK of the different OM formulations by comparing test treatment groups to the reference group for AUCₗₐₛₜ, AUC_{inf}, and Cₘₐₓ. Treatment comparisons were as follows with Treatment Group A being the reference in both instances:

1. OM, 25×1 mg oral slow-release mini-tablets (Treatment Group B) versus OM, 1×25 mg oral MR tablet (Treatment Group A)

2. OM, 25×1 mg oral fast-release mini-tablets (Treatment Group C) versus OM, 1×25 mg oral MR tablet (Treatment Group A) respectively.

The natural log (In) transformed PK parameters were analyzed using a mixed model. The model included treatment and sequence as fixed effect and subject nested within sequence as a random effect.

For each PK parameter separately, the least squares mean (LSM) for each treatment group, difference in LSMs between the test and reference treatment groups, and corresponding 90% confidence interval (CI) were calculated; these values were then back transformed to give the geometric LSM (GLSM), ratio of GLSMs, and corresponding 90% CI.

Additionally, the pooled estimate (across all treatment groups) of the within-subject coefficient of variation (CV) was calculated, and residual plots were produced to assess the adequacy of the model(s) fitted.

All safety data were listed. The treatment-emergent adverse events were summarized by treatment, severity, and relationship to the study drug. The frequency of treatment emergent adverse events was summarized by treatment, system organ class and preferred term of the Medical Dictionary for Regulatory Activities. No inferential statistical analyses were planned.

The results of the study are summarized in Tables 8 and 9.

TABLE 8
Summary of the Plasma Pharmacokinetic Parameter Estimates for OM (n = 20)

| Parameter | Group A | Group B | Group C | Group D¹ | Group E |
|---|---|---|---|---|---|
| AUCₗₐₛₜ (h*ng/mL) (SD) | 2030 (31.7) | 2020 (35.7) | 2550 (25.8) | 430 (30.9) | 564 (26.7) |
| AUC_{inf} (h*ng/mL) (SD) | 2080 (31.1) | 2080 (34.0) | 2600 (25.3) | 478 (27.6) | 612 (24.4) |
| Cₘₐₓ (ng/mL) (SD) | 52.4 (27.2) | 67.4 (39.5) | 116 (29.8) | 16.1 (36.2) | 27.2 (20.1) |
| tₘₐₓ (h) (min-max) | 4.0 (1.5-12.0) | 3.0 (2.0-8.0) | 2.0 (1.5-4.0) | 4.0 (2.0-6.0) | 2.0 (1.0-4.1) |
| tₗₐₛₜ (h) (min-max) | 132 (96.0-168) | 144 (72.0-168) | 144 (72.0-168) | 72.0 (48.0-96.0) | 72.0 (48.0-120) |
| t_{1/2} (h) (SD) | 22.7 (3.4) | 24.5 (4.2) | 23.5 (4.7) | 23.0 (3.4) | 22.0 (4.0) |

¹n = 17

TABLE 9
Summary of the Statistical Analysis¹ of Plasma Pharmacokinetic Parameter Estimates for OM (n = 20)

| | GLSM | GLSM | Ratio B:A | |
|---|---|---|---|---|
| Parameter | Group A | Group B | (90% Cl) | CV¹ |
| AUCₗₐₛₜ (h*ng/mL) (SD) | 2030 (31.7) | 2020 (35.7) | 1.0 (0.93, 1.1) | 12.5 |
| AUC_{inf} (h*ng/mL) (SD) | 2080 (31.1) | 2080 (34.0) | 1.0 (0.94, 1.1) | 11.9 |
| Cₘₐₓ (ng/mL) (SD) | 52.4 (27.2) | 67.4 (39.5) | 1.3 (1.2, 1.4) | 20.3 |

1Model: In (parameters) = treatment + sequence + random error; the ratios and CIs were obtained by taking the exponential of the corresponding differences and CIs on the natural-log (In) scale.
²CV refers to “coefficient of variation”

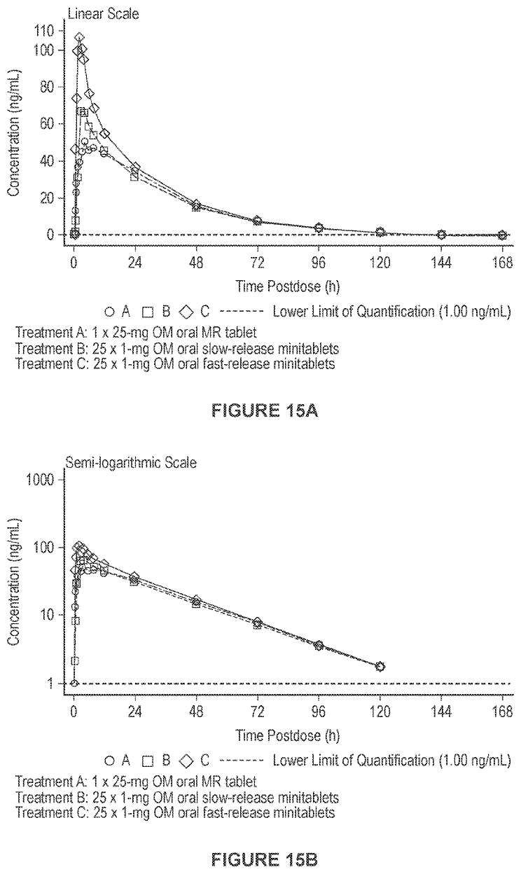

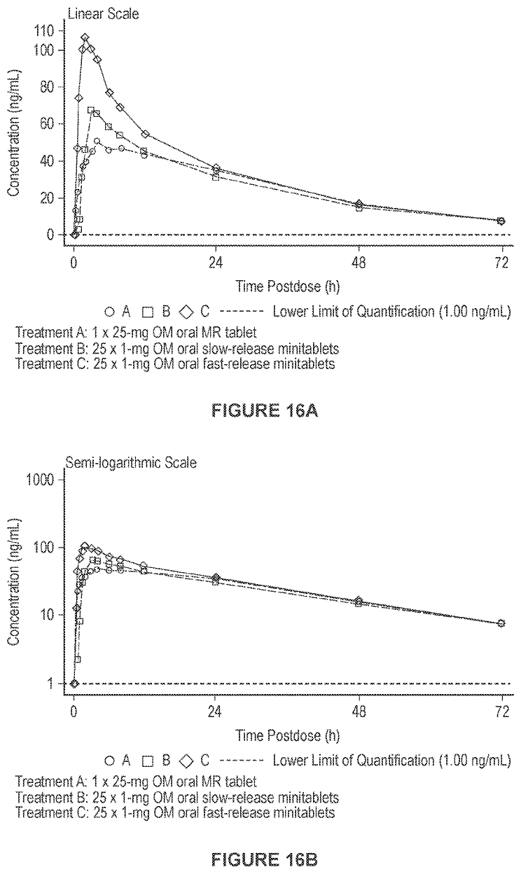

As shown in Tables 8 and 9, following administration of 25×1 mg OM slow-release mini-tablets, median OM tₘₐₓ occurred earlier compared to administration of a single 25 mg OM MR tablet, although the range of tₘₐₓ values was similar. Geometric mean AUCₗₐₛₜ and AUC_{inf} values were similar between the 2 treatment groups, while Cₘₐₓ was higher following administration of 25×1 mg OM slow-release mini-tablets compared to a single 25 mg OM MR tablet. Arithmetic mean t_{1/2} values for OM were similar between the 2 treatment groups (Table 8 and FIGS. 15A-16B). The ratios (test/reference groups) of the GLSM of 25×1 mg OM slow-release mini-tablets compared to a single 25 mg OM MR tablet were 0.9980, 0.9998, and 1.2858 for AUCₗₐₛₜ, AUC_{inf}, and Cₘₐₓ, respectively (Table 9).

Following administration of 25×1 mg OM fast-release mini-tablets, median OM tₘₐₓ occurred earlier compared to administration of a single 25 mg OM MR tablet. Exposure, based on geometric mean AUCs and Cₘₐₓ, was higher following administration of 25 mg OM fast-release mini-tablets compared to administration of a single 25 mg OM MR tablet. Arithmetic mean t_{1/2} values for OM were similar between the 2 treatment groups (Table 8 and FIGS. 15A-16B). The ratios (test/reference) of the GLSM of 25×1 mg OM fast-release mini-tablets compared to a single 25 mg OM MR tablet were 1.2560, 1.2503, and 2.2136 for AUCₗₐₛₜ, AUC_{inf}, and Cₘₐₓ, respectively (Table 9).

As shown in Table 8, following administration of 6×1 mg OM slow-release mini-tablets, median OM tₘₐₓ occurred 2 hours later compared to administration of 6×1 mg OM fast release mini-tablets. Exposure, based on geometric mean AUCs and Cₘₐₓ, was higher following administration of the 6 mg OM fast-release mini-tablet dose compared to the 6 mg OM slow-release mini-tablet dose. Arithmetic mean t_{1/2} values for OM were similar between the 2 treatment groups.

As demonstrated by the data shown in Tables 8 and 9, when OM was administered as 25×1 mg OM slow-release mini-tablets, AUCₗₐₛₜ, AUC_{inf}, and Cₘₐₓ were 0.9980-, 0.9998-, and 1.2858-fold to that of a single 25 mg OM MR tablet, respectively. Also, when OM was administered as 25×1 mg OM fast-release mini-tablets, AUCₗₐₛₜ, AUC_{inf}, and Cₘₐₓ were 1.2560-, 1.2503-, and 2.2136-fold to that a single 25 mg OM MR tablet, respectively.

Single oral doses of OM were safe and well tolerated by healthy adult subjects when administered as 25 mg (1×25 mg) MR tablet formulation, 25 mg (25×1 mg) slow-release mini-tablet formulation, 25 mg (25×1 mg) fast release mini-tablet formulation, 6 mg (6×1 mg) slow-release mini-tablet formulation, and 6 mg (6×1 mg) fast-release mini-tablet formulation.

All treatment-emergent adverse events were mild in severity and resolved by the end of the study. There were no serious adverse events or treatment-emergent adverse events leading to discontinuation from the study.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (for example, “such as”) provided herein, is intended merely to better illuminate the invention and does not pose a limitation on the scope of the invention unless otherwise claimed. No language in the specification should be construed as indicating any non-claimed element as essential to the practice of the invention.

What is claimed:

1. A tablet formulation comprising:
a core comprising
omecamtiv mecarbil, a pharmaceutically acceptable salt thereof, or a pharmaceutically acceptable hydrate of a pharmaceutically acceptable salt thereof;
a filler;
a binder;
a glidant; and
a lubricant; and
a film coating on the core, the film coating comprising
a modified-release polymer and a pore former.

2. The tablet formulation of claim 1, wherein the omecamtiv mecarbil is present as omecamtiv mecarbil dihydrochloride monohydrate.

3. The tablet formulation of claim 1 or 2, wherein the filler comprises microcrystalline cellulose, lactose monohydrate, or a combination thereof.

4. The tablet formulation of claim 3, wherein the filler comprises microcrystalline cellulose and lactose monohydrate.

5. The tablet formulation of any one of claims 1 to 4, wherein the binder comprises hydroxypropyl cellulose.

6. The tablet formulation of any one of claims 1 to 5, wherein the glidant comprises silicon dioxide.

7. The tablet formulation of any one of claims 1 to 6, wherein the lubricant comprises magnesium stearate.

8. The tablet formulation of any one of claims 1 to 7, wherein the core comprises
8-11 wt. % omecamtiv mecarbil, a pharmaceutically acceptable salt thereof, or a pharmaceutically acceptable hydrate of a pharmaceutically acceptable salt thereof;
83-86 wt. % filler;
2-5 wt. % binder;
0.2-0.8 wt. % glidant; and
0.8-1.2 wt. % lubricant.

9. The tablet formulation of any one of claims 1 to 8, wherein the film coating further comprises a plasticizer.

10. The tablet formulation of any one of claims 1 to 9, wherein the pore former is also a plasticizer.

11. The tablet formulation of any one of claims 1 to 10, wherein the modified-release polymer of the film coating comprises ethylcellulose, poly(ethyl acrylate-co-methylmethacrylate), poly(ethyl acrylate-co-methylmethacrylate-co-trimethylammonioethylmethacrylate chloride), cellulose acetate, polyvinyl acetate, or a combination thereof.

12. The tablet formulation of claim 11, wherein the modified-release polymer comprises cellulose acetate.

13. The tablet formulation of any one of claims 1 to 12, wherein the pore former of the film coating comprises hypromellose, polyvinylpyrrolidone, sorbitol, triethyl citrate, polyethylene glycol, or a combination thereof.

14. The tablet formulation of claim 13, wherein the pore former comprises polyethylene glycol.

15. The tablet formulation of claim 14, wherein the polyethylene glycol is polyethylene glycol 3350.

16. The tablet formulation of any one of claims 9 to 15, wherein the plasticizer comprises polyethylene glycol, diethyl phthalate, triethyl citrate, dibutyl sebacate, triacetin, or a combination thereof.

17. The tablet formulation of any one of claims 11 to 16, wherein the film coating comprises
50-90 wt. % modified-release polymer; and
10-50 wt. % pore former and plasticizer (when present).

18. The tablet formulation of claim 17, wherein the film coating comprises 60 wt. % modified-release polymer and 40 wt. % pore former and plasticizer (when present).

19. The tablet formulation of any one of claims 1 to 18, wherein the film coating comprises 9 wt. % of the total weight of the tablet formulation.

20. The tablet formulation of any one of claims 1 to 18, wherein the film coating comprises 13 wt. % of the total weight of the tablet formulation.

21. The tablet formulation of any one of claims 1 to 18, wherein the film coating comprises 23 wt. % of the total weight of the tablet formulation.

22. The tablet formulation of claim 1, comprising:
5-40 wt. % omecamtiv mecarbil dihydrochloride monohydrate;
10-45 wt. % microcrystalline cellulose;
10-45 wt. % lactose monohydrate;
1-8 wt. % hydroxypropyl cellulose;
0.1-2 wt. % colloidal silicon dioxide;
0.25-3 wt. % magnesium stearate;
3-20 wt. % cellulose acetate; and
2-15 wt. % polyethylene glycol.

23. The tablet formulation of claim 22, comprising:
5-10 wt. % omecamtiv mecarbil dihydrochloride monohydrate;
30-45 wt. % microcrystalline cellulose;
30-45 wt. % lactose monohydrate;
1-5 wt. % hydroxypropyl cellulose;
0.1-2 wt. % colloidal silicon dioxide;
0.5-3 wt. % magnesium stearate;
3-20 wt. % cellulose acetate; and
2-15 wt. % polyethylene glycol.

24. The tablet formulation of claim 23, comprising:
9 wt. % omecamtiv mecarbil dihydrochloride monohydrate;
38.9 wt. % microcrystalline cellulose;
38.9 wt. % lactose monohydrate;
2.7 wt. % hydroxypropyl cellulose;
0.5 wt. % colloidal silicon dioxide;
1 wt. % magnesium stearate;
5.4 wt. % cellulose acetate; and
3.6 wt. % polyethylene glycol.

25. The tablet formulation of claim 23, comprising:
8.5 wt. % omecamtiv mecarbil dihydrochloride monohydrate;
37.3 wt. % microcrystalline cellulose;
37.3 wt. % lactose monohydrate;
2.6 wt. % hydroxypropyl cellulose;
0.4 wt. % colloidal silicon dioxide;
0.9 wt. % magnesium stearate;
7.8 wt. % cellulose acetate; and
5.2 wt. % polyethylene glycol.

26. The tablet formulation of claim 23, comprising:
7.5 wt. % omecamtiv mecarbil dihydrochloride monohydrate;
33 wt. % microcrystalline cellulose;
33 wt. % lactose monohydrate;
2.3 wt. % hydroxypropyl cellulose;
0.4 wt. % colloidal silicon dioxide;
0.8 wt. % magnesium stearate;
13.8 wt. % cellulose acetate; and
9.2 wt. % polyethylene glycol.

27. The tablet formulation of claim 1, wherein
the core comprises
9.8 wt. % omecamtiv mecarbil dihydrochloride monohydrate;
42.8 wt. % microcrystalline cellulose;
42.8 wt. % lactose monohydrate;
3 wt. % hydroxpropyl cellulose;
0.5 wt. % colloidal silicon dioxide; and
1 wt. % magnesium stearate; and wherein
the film coating results in a 10% coating weight gain for the tablet formulation based upon core total weight, and wherein the film coating comprises 60 wt. % cellulose acetate and 40 wt. % polyethylene glycol, based upon total weight of the film coating.

28. The tablet formulation of claim 1, wherein
the core comprises
9.8 wt. % omecamtiv mecarbil dihydrochloride monohydrate;
42.8 wt. % microcrystalline cellulose;
42.8 wt. % lactose monohydrate;
3 wt. % hydroxpropyl cellulose;
0.5 wt. % colloidal silicon dioxide; and
1 wt. % magnesium stearate; and wherein
the film coating results in a 15% coating weight gain for the tablet formulation based upon core total weight, and wherein the film coating comprises 60 wt. % cellulose acetate and 40 wt. % polyethylene glycol, based upon total weight of the film coating.

29. The tablet formulation of claim 1, wherein
the core comprises
9.8 wt. % omecamtiv mecarbil dihydrochloride monohydrate;
42.8 wt. % microcrystalline cellulose;
42.8 wt. % lactose monohydrate;
3 wt. % hydroxpropyl cellulose;
0.5 wt. % colloidal silicon dioxide; and
1 wt. % magnesium stearate; and wherein
the film coating results in a 30% coating weight gain for the tablet formulation based upon core total weight, and wherein the film coating comprises 60 wt. % cellulose acetate and 40 wt. % polyethylene glycol, based upon total weight of the film coating.

30. The tablet formulation of any one of claims 1 to 29, comprising 1-3 mg omecamtiv mecarbil.

31. The tablet formulation of claim 30, comprising 1 mg omecamtiv mecarbil.

32. The tablet formulation of any one of claims 1 to 24, 27, 30, and 31 having an omecamtiv mecarbil release profile of:
less than or equal to 50% omecamtiv mecarbil released at 1 hour;
60-70% omecamtiv mecarbil released at 2 hours;
85-90% omecamtiv mecarbil released at 8 hours; and
greater than or equal to 90% omecamtiv mecarbil released at 16 hours.

33. The tablet formulation of any one of claims 1 to 23, 25, 28, 30, and 31 having an omecamtiv mecarbil release profile of:
less than or equal to 25% omecamtiv mecarbil released at 1 hour;
35-45% omecamtiv mecarbil released at 2 hours;
75-80% omecamtiv mecarbil released at 8 hours; and
greater than or equal to 85% omecamtiv mecarbil released at 16 hours.

34. The tablet formulation of any one of claims 1 to 23, 26, and 29 to 31, having an omecamtiv mecarbil release profile of:
less than or equal to 10% omecamtiv mecarbil released at 1 hour;
25-35% omecamtiv mecarbil released at 2 hours;
70-75% omecamtiv mecarbil released at 8 hours; and
greater than or equal to 78% omecamtiv mecarbil released at 16 hours.

35. The tablet formulation of any one of claims 1-34, wherein upon administration to a patient, the tablet formulation provides a maximum plasma concentration (Cₘₐₓ) of omecamtiv mecarbil in the patient of 100-1000 ng/mL

36. The tablet formulation of any one of claims 1 to 35, not comprising (free of) a pH-modifying agent.

37. The tablet formulation of any one of claims 1 to 36 having a diameter of up to 3 mm.

38. A method of treating heart failure in a patient suffering therefrom, comprising administering to the patient the tablet formulation of any one of claims 1 to 37.

39. The method of claim 38, wherein the heart failure is acute or chronic.

40. The method of claim 38, wherein the heart failure is heart failure with reduced ejection fraction (HFrEF).

41. The method of any one of claims 38 to 40, wherein the patient is a pediatric patient.

42. The method of claim 41, wherein the pediatric patient is administered the tablet formulation in an amount to provide 3-25 mg twice daily omecamtiv mecarbil.

43. The method of any one of claims 38 to 40, wherein the patient is an adult patient with difficulty swallowing.

44. The method of claim 43, wherein the adult patient is administered the tablet formulation in an amount to provide 25 mg or 50 mg twice daily omecamtiv mecarbil.

45. The tablet formulation of any one of claims 1 to 37 for use in treating heart failure.

46. The tablet formulation of claim 45, wherein the heart failure is acute or chronic.

47. The tablet formulation of claim 45, wherein the heart failure is heart failure with reduced ejection fraction (HFrEF).

48. The tablet formulation of any one of claims 45 to 47, wherein the tablet formulation is suitable for administration to a pediatric patient.

49. The tablet formulation of claim 48, wherein the pediatric patient is administered the tablet formulation in an amount to provide 3-25 mg twice daily omecamtiv mecarbil.

50. The tablet formulation of any one of claims 45 to 47, wherein the tablet formulation is suitable for administration to an adult patient with difficulty swallowing.

51. The tablet formulation of claim 50, wherein the adult patient is administered the tablet formulation in an amount to provide 25 mg or 50 mg twice daily omecamtiv mecarbil.

52. Use of the tablet formulation of any one of claims 1 to 37 in the preparation of a medicament for the treatment of heart failure.

53. The use of claim 52, wherein the heart failure is acute or chronic.

54. The use of claim 52, wherein the heart failure is heart failure with reduced ejection fraction (HFrEF).

55. The use of any one of claims 52 to 54, wherein the tablet formulation is suitable for administration to a pediatric patient.

56. The use of claim 55, wherein the pediatric patient is administered the tablet formulation in an amount to provide 3-25 mg twice daily omecamtiv mecarbil.

57. The use of any one of claims 52 to 54, wherein the tablet formulation is suitable for administration to an adult patient with difficulty swallowing.

58. The use of claim 57, wherein the adult patient is administered the tablet formulation in an amount to provide 25 mg or 50 mg twice daily omecamtiv mecarbil.

59. A process for making the tablet formulation of any one of claims 1 to 37, comprising:
admixing the omecamtiv mecarbil, a pharmaceutically acceptable salt thereof, or a pharmaceutically acceptable hydrate of a pharmaceutically acceptable salt thereof, the filler, and the binder and granulating to form a granulated mixture;
admixing the granulated mixture and a granulating solvent, and granulating to form a wet granulate;
drying the wet granulate to form a dried granulate;
milling the dried granulate to form a milled granulate;
admixing the milled granulate, the glidant, and the lubricant and compressing the admixture to form the core;
admixing the core with a film coating pre-mixture to provide the film coating over the core, and
drying the coated core to form the tablet formulation,
wherein the film coating pre-mixture comprises the modified-release polymer, the plasticizer, and a film coating solvent.

60. The process of claim 59, wherein the admixing of the omecamtiv mecarbil, a pharmaceutically acceptable salt thereof, or a pharmaceutically acceptable hydrate of a pharmaceutically acceptable salt thereof, the filler, and binder is performed with a high shear granulator.

61. The process of claim 59 or 60, wherein the granulating solvent comprises water.

62. The process of any one of claims 59 to 61, wherein the milling of the dried granulate is performed using an impact mill.

63. The process of any one of claims 59 to 62, wherein the admixing of the milled granulate, the glidant, and the lubricant is performed stepwise such that the milled granulate and the glidant are admixed, then the lubricant is admixed with the resulting mixture.

64. The process of any one of claims 59 to 63, wherein the film coating solvent comprises acetone, water, or a mixture thereof.

65. The process of claim 64, wherein the film coating solvent comprises acetone and water.

66. The process of claim 65, wherein the film coating solvent comprises 9:1 acetone:water.

67. The process of any one of claims 59 to 66, wherein the admixing of the core and the film coating pre-mixture is performed in a fluid bed coater.

68. A process of making the tablet formulation of any one of claims 1 to 37, comprising
admixing the omecamtiv mecarbil, a pharmaceutically acceptable salt thereof, or a pharmaceutically acceptable hydrate of a pharmaceutically acceptable salt thereof, the filler, and the binder and granulating to form a granulated mixture;
milling the granulated mixture to form a milled granulate;
admixing the milled granulate, the glidant, and the lubricant and compressing the admixture to form the core;
admixing the core with a film coating pre-mixture to provide the film coating over the core, and
drying the coated core to form the tablet formulation,
wherein the film coating pre-mixture comprises the modified-release polymer, the plasticizer (when present), and a film coating solvent.

69. A process of making the tablet formulation of any one of claims 1 to 37, comprising
admixing the omecamtiv mecarbil, a pharmaceutically acceptable salt thereof, or a pharmaceutically acceptable hydrate of a pharmaceutically acceptable salt thereof, the filler, the binder, the glidant, and the lubricant and compressing the admixture to form the core;
admixing the core with a film coating pre-mixture to provide the film coating over the core, and
drying the coated core to form the tablet formulation,
wherein the film coating pre-mixture comprises the modified-release polymer, the plasticizer (when present), and a film coating solvent.
